(19) United States
(12) Patent Application Publication
(10) Pub. No.: US 2003/0225392 A1
(43) Pub. Date: Dec. 4, 2003

(54) Low profile transpyloric jejunostomy system and method to enable

(75) Inventors: Donald J. McMichael, South Jordan, UT (US); Mark Elliott Foster, Sandy, UT (US); Dennis Cox, Sandy, UT (US); Kelly Jay Christian, Draper, UT (US)

Correspondence Address: KIMBERLY-CLARK WORLDWIDE, INC., 401 NORTH LAKE STREET, NEENAH, WI, 54956

(73) Assignee: Kimberly-Clark Worldwide, Inc.

(21) Appl. No.: 10/159,515

(22) Filed: May 31, 2002

(51) Int. Cl. A61M 31/00

(52) U.S. Cl. 604/509; 604/270

(57) ABSTRACT

The present invention is directed to methods for inserting or placing the low profile catheter or system in a patient. The apparatus of the present invention is designed such that it may be used both as an original device as well as a replacement device. The device is also capable of being placed or inserted in a patient in a variety of ways, including, for example, those which are highly invasive, such as those placements or insertions which occur during a surgical procedure, as well as those procedures which are minimally invasive and/or those which produce or result in little or no blood loss and which may or may not require a surgeon to perform. The present invention is intended to cover all such procedures as discussed herein.

### BACKGROUND OF THE INVENTION

The present invention relates generally to enteral tubes, and more particularly to a low profile transpyloric jejunostomy balloon catheter having a low profile and methods to enable placement of the low profile devices.

There are numerous situations in which a body cavity must be catheterized to achieve a desired medical goal. One relatively common situation in which a body cavity is catheterized is to provide nutritional solutions or medicines directly into the stomach or intestines. A stoma is formed in the stomach or intestinal wall and a catheter is placed through the stoma. Feeding solutions can be injected through the catheter to provide nutrients directly to the stomach or intestines (known as enteral feeding). Additionally, with the use of enteral feeding catheters, for example, it is generally necessary to ensure that the catheter is not accidentally dislodged or removed from the stomach or intestines. This is true both during the actual administration or removal of fluids as well as the time periods in between feedings.

To ensure that the catheter is maintained in the proper position, it is common to use a balloon disposed along the catheter shaft. Inflating the balloon causes the balloon to contact the anatomical structure (i.e., a duct or stomach wall) and thereby prevent the catheter from moving out of the proper position. Depending on the type of catheter, the balloon may be positioned in a variety of locations along the catheter shaft. For example, with a G-tube the balloon will generally be at or near the distal end of the catheter, although the balloon or other retention mechanism may be slightly closer to the head of the catheter provided that the retention effect may still be achieved. Such balloon catheter devices may include a “low-profile” head at the proximal end of the catheter shaft. The head, which may help hold the balloon catheter in place, includes an opening for receiving the feeding solution and a one-way valve for preventing fluids from passing out of the patient via the catheter. U.S. Pat. Nos. 5,997,503 and 5,997,546, both owned by Applicants' Assignee and incorporated in their entirety by reference herein, disclose examples of low-profile balloon catheters suitable for enteral feeding.

As indicated above, there are a variety of instances in which it may be necessary to use a catheter, one of which is the not uncommon reaction following major surgery for one's stomach function to be impaired for a period of time. For instance, in addition to the need to supply or supplement the body with a certain level of nutrients and the like, following surgery, after an accident, as well as in other instances of impaired or limited gastric functionality, an unfed gut can become a source of bacteria that gets into the bloodstream. These types of problems may be resolved by the introduction of nutrients through a jejuneal tube properly inserted through the abdominal wall, gastric wall, pylorous, duodenum of a patient, into the jejunum beyond the Ligament of Treitz.

Transpyloric passage of a jejunal tube is technically difficult to achieve because of at least the following anatomic problems. First, the pylorus and duodenum are in the retro-peritoneum covered by small bowel mesentery and the right colon, making them poorly accessible to the surgeon's fingers. Secondly, the mucosal folds of the duodenum is redundant and creates ridges that prevent easy passage of a catheter.

While prior devices that are designed to span the pylorus are known to exist, each have certain shortcomings. For example, the Moss dual lumen gastrostomy tube, as described in Moss, U.S. Pat. Nos. 4,543,089 and 4,642,092, is intended for transpyloric feeding; however, the tube does not go beyond the duodenum. Since the tip of the Moss tube is situated just beyond the pylorus, nutrients from the tube tend to go back to the stomach rather than moving on to the jejunum. Accordingly, the Moss tube has proved of little benefit to critically ill patients who have had major surgical illness.

The Nyhus-Nelson system is a tube with two balloons that allow the surgeon to do a “push-me/pull-me” technique of passing a tube through the duodenum. This system has the disadvantage of being very time consuming for the surgeon and having a relatively high rate of failure.

Cook Incorporated markets a Carey-Alzate-Coons double lumen gastrojejunostomy set, as described in U.S. Pat. Nos. 4,581,025 and Re. 31,855, wherein the catheter is advanced over a wire guide after insertion of the wire guide through the pylorus and into the duodenum. The Cook device is designed for placement by a radiologist or gastroenterologist with access to an x-ray machine but is not suited for use at the operating table by a surgeon during major abdominal surgery.

Referring to the illustrative drawing of FIG. 1, there is shown a perspective view of an earlier enteral feeding device 20. The device 20 includes an elongated tubular member 51 formed from a stretchable elastomeric material such as silicone. FIG. 1A is an illustrative cross-sectional view of the tubular member 51 of the earlier device. The member 51 defines a jejunal tube 22, a gastronony tube 34 and a fluid line 46.

The jejunal feeding tube 22 includes an outlet end portion 24 which can extend through a patients stomach into the jejunum. The jejunal tube outlet end portion includes perforations 26 which permit liquid food or medication to pass therethrough. The tube 22 is integrally connected to a jejunal tube inlet end portion 28 which defines a jejunal inlet port 30 having a removable plug cover 32.

The gastrostomy tube 34 is shorter than the jejunal tube 22 and includes a plurality of drainage inlets or food outlet ports such as inlet/outlet 36. A gastrostomy tube end portion 37 defines a gastrostomy inlet port 38 having a plug cover 40.

An inflatable balloon 42 is provided near the end of the gastrostomy tube 34 and is inflatable through a valve 44. The valve 44 is used to supply fluid to the balloon 42 through the fluid line 46.

Frictional contact between the elongated tubular member 51 and a locking/retention ring 56 is sufficiently great to prevent the member 51 from moving further into the stomach. The locking ring 56 remains in contact with a patient's abdominal wall during use. However, the frictional contact also is sufficiently low to permit adjustment or placement of the member 51 relative to a patients abdomen.

Referring to the illustrative drawings of FIG. 2, there is shown a perspective view of an earlier device 20 in use. The inflated balloon 42 forms a gasket that seals the entrance to the stomach, and together with the locking ring 56 secures the device 20 in place.

While prior feeding tubes generally have been able to accomplish the desired task of providing nutrition to a patient, there have been shortcomings with their use. In particular, transpyloric jejunal (also referred to as gastrojejunal) tubes were originally designed for use with non-ambulatory patients. As such, it was not previously important how long or far the tube or lumens extended from or out from the patient. Previously, it was not uncommon for large external quantities of tubing to be present. However, as experienced with a number of different patients, for example, combative or ambulatory patients, the length of tubing which extends from the patient was deemed not only unnecessary, but detrimental as it has a tendency to get tangled or caught on something. In such instances the chance of dislodging the tube or rendering the tube ineffective was high, and thereby significantly increased the chance that the tube insertion procedure would need to be repeated as well as the possibility that the tube could cause damage when the dislodged. Additionally in ambulatory patients, there is the potential for cosmetic embarrassment, in that it was previously difficult to hide the presence of the tubing from others when access to the tubing was not necessary or required. Even when the patient is non-ambulatory, in some situations (e.g. children and/or patients prone to seizure), it would be desirable to have the option of a low profile tube.

Attempts have been made to provide a low-profile feeding tube, such as U.S. Pat. No. 5,073,166 to Parks et al. and U.S. Pat. No. 6,264,631 to Willis et al., both of which are commonly owned by the Assignee of the present invention, and both of which are incorporated by reference in their entirety. However, these devices are not intended for transpyloric use. WO 01/60313, assigned to Sherwood Services, also makes an attempt to address some of the concerns with non-low-profile feeding devices, via the disclosure of an adapter and a system which uses the adapter; however, devices disclosed therein require the person inserting the adapter to feed a feeding tube through the adapter before the adapter can be used and to undertake several other preparation steps (some post insertion) before the adapter or the system could be considered low profile.

Thus, while a number of improvements have been made to conventional enteral tubes, there exists the need and the desire to produce a low-profile TJ tube or system which is complete and/or does not require on-site assembly or point of use construction. Generally, the users of TJ tubes would also benefit (e.g. reduced dislodgements and/or improvement in the ability to reduce cosmetic embarrassment and the like) from a low-profile article.

### SUMMARY OF THE INVENTION

In response to the difficulties and problems discussed above, a new low profile catheter has been developed. The present invention relates to a catheter or system for providing nutrition and/or medication to a patient whose stomach function is inhibited or nonfunctional. More particularly, the present invention relates to feeding tubes or feeding devices, such as transpyloric feeding tubes and the like, which are adapted to enable the provision of nutrition and/or medicine directly into the patient's jejunum where the patient is experiencing a nonfunctioning or impaired stomach and/or difficulties swallowing, chewing or the like. The devices contemplated by the present invention may also permit nutrients to be placed directly into a patient's stomach and/or into the patients jejunum. As noted above, this may be necessary when a patient has a disorder of the gastrointestinal tract, malabsorption (impaired absorption of nutrients, vitamins or minerals from the diet by the lining of the small intestine), or neurological or renal disorders.

More specifically one aspect of the present invention relates to a catheter having a head, a catheter segment, and a retention mechanism. The head of the catheter has at least two openings through which fluid (including gases) may pass to or from a patient, the head having a low profile relative to a patient when the catheter is properly positioned in the patient. The catheter segment extends from the head of the catheter and has a proximal end and a distal end. The catheter has at least a first and second lumen, each lumen being in communication with at least one of the openings of the head. The openings in the head of the catheter are designed to allow for the passage of fluid, including fluids with solids, into the catheter and/or out of the patient. The low profile head may contribute to a reduction in the number of accidental removals or displacements of the feeding tubes or systems as compared to traditional “non-low profile” apparatus in that the amount or length of the catheter or system which extends external the patient's body is significantly less. Additionally, the improved cosmetic appearance of a low profile system or device is desirable for many patents, especially those who are ambulatory and/or those individuals who would like to be able to conceal the fact that they have an enteral feeding tube.

In one of the embodiments of the present invention, the retention mechanism may be a balloon. Specifically, the balloon may be formed by a sleeve with a first end attached to the catheter segment so as to form a first cuff and a second end attached to the catheter segment so as to form a second cuff, the balloon collapsing on the catheter segment when not inflated.

For purposes of this application, unless the context demands a different meaning, or a different meaning is expressed, the term “stoma” is intended to include fully formed stomas (as will be appreciated by those in the art) as well as any other opening or aperture or the like (including, for example, punctures, holes, passages or the like through, for example, the abdominal wall or gastric wall of a patient), even if newly created and/or not fully formed or healed (as with conventional stomas), through which the device of the present invention may be passed in accordance with the description herein so as to enable the uses contemplated by the present invention. References to the passage of an item through the abdominal or gastric wall of a patient is intended to include not only initial insertions or the creation of an opening, puncture, or the like so as to enable the passage of the item therethrough, but also passage of the item or items through a existing opening (e.g. an existing puncture or an existing stoma). As used herein, unless the context demands a different meaning, or a different meaning is expressed, the term “patient” is intended to include any and all such patients and is not limited to humans, but may also include, for example, other creatures such as animals, mammals and the like. As used herein, unless the context demands a different meaning, or a different meaning is expressed, the terms “puncture” or “puncturing” when used as a verb is intended to include all manners of creating an opening, hole, aperture or the like, including, but not limited to puncturing, piercing, cutting, slitting and the like. As used herein, unless the context demands a different meaning, or a different meaning is expressed, the term “feed tube” means any and all suitable devices which may be used in accordance with the present invention to provide food, nutrition, medicine, fluids and/or the like therethrough. An example of a suitable “feed tube” is a catheter, and more specifically a transjejunal catheter.

Another embodiment of the present invention relates to a balloon catheter adapted for placement through a stoma into a body cavity so that the balloon catheter is maintained in the stoma. The balloon catheter includes a head having at least two openings through which a fluid may be introduced or passed, the head being low profile relative to a patient when the catheter is correctly positioned within the patient; a catheter segment extending from the head to a distal tip of the catheter, the catheter segment having an exterior and a wall defining at least one passageway through an interior; and an elongate sleeve attached to the exterior of the catheter segment about the passageway so as to form an inflatable balloon which covers a portion of the catheter segment. The sleeve may preferably be formed such that in its collapsed state (i.e. when not inflated) the sleeve closely surrounds the catheter segment, and when inflated the sleeve extends radially outwardly from the catheter segment. In this embodiment, the inflation of the sleeve may be controlled through one of the openings in the head of catheter as the sleeve is in communication with one of the openings in the head of the balloon catheter.

Yet another embodiment of the present invention defines a balloon catheter adapted for placement through a stoma into a body cavity so that the balloon catheter is maintained in the stoma. The balloon catheter includes a low profile head having at least three openings through which a fluid may be injected, the head having a low profile relative to a patient's body when the catheter is correctly positioned; a catheter segment extending from the head to a distal tip, the catheter segment having an exterior and a wall defining a passageway through the interior, the passageway defining at least three lumens, each lumen having a proximal end and a distal end; and an elongate sleeve attached to the catheter segment about the passageway so as to form an inflatable balloon which covers a portion of the exterior of the catheter segment, the elongate sleeve being in communication with one of the at least three lumens, the sleeve being in collapsed state when not inflated wherein the sleeve closely surrounds the catheter segment, and wherein the sleeve extends radially outwardly from the catheter segment when inflated.

One embodiment of the present invention is also directed to a low profile transjejunal feeding system. The feeding system includes a balloon catheter having multiple lumens, a low profile head having at least two ports therein, and a balloon retention mechanism; and a first adapter capable of attachment to one of the ports of the balloon catheter; wherein each of the lumens is in communication with at least one of the ports.

The present invention is also directed to methods for inserting or placing the low profile catheter or system in a patient. The apparatus of the present invention is designed such that it may be used both as an original device as well as a replacement device. The device is also capable of being placed in a patient in a variety of ways, including, for example, those which are highly invasive, such as those placements or insertions which occur during a surgical procedure, as well as those procedures which are minimally invasive and/or those which produce or result in little or no blood loss and which may or may not require a surgeon to perform. The present invention is intended to cover all such procedures. Specifically, one embodiment of the present invention is directed to a method for placement of an elongated low profile flexible feed tube through the abdominal wall, gastric wall, pylorus and duodenum of a patient into the jejunum beyond the Ligament of Treitz. The method includes providing an abdominal wall trocar, an elongated tubular sheath of a size and shape to closely conform to the trocar, an elongated tubular gastrostomy introducer having a tapered free end with an inlet opening therethrough, an elongated slotted cannulator having a longitudinal slot and internal width to accommodate passage of the introducer therethrough, and an elongated flexible feed tube. The cannulator is preferably insertable through the sheath. The method further includes telescopically sliding the sheath onto the trocar, thrusting the trocar and sheath together through the abdominal wall to form a puncture wound therethrough, removing the trocar from the sheath, puncturing the gastric wall at a position for alignment with the puncture wound in the abdominal wall, inserting the gastrostomy introducer through the gastric wall puncture into the lumen of the stomach, aspirating the stomach through the introducer, fitting the slotted cannulator onto the introducer and passing the cannulator through the gastric wall puncture into the stomach, withdrawing the introducer from the stomach through the cannulator, manipulating the cannulator externally of the stomach to move the end of the cannulator within the stomach through the pylorus of the patient, inserting the feed tube through the sheath in the abdominal wall, inserting the feed tube into the cannulator and advancing the distal end of the feed tube through the stomach, pylorus and duodenum into the jejunum beyond the Ligament of Treitz, and withdrawing the cannulator through the puncture whereby the feed tube is maintained in its inserted position. The step of withdrawing the cannulator through the puncture such that the tube is maintained in its inserted position may preferably be achieved by peeling or otherwise removing the feed tube from the cannulator through the longitudinal slot therein.

As above, the present invention is also directed to alternate methods of placing the devices of the present invention, those methods are set forth in more detail in the detailed description below.

The invention will be more fully understood and further advantages will become apparent when reference is made to the following detailed description of the invention and the accompanying drawings.

### BRIEF DESCRIPTION OF THE DRAWINGS

FIG. 1 is a perspective view of an earlier feeding tube.

FIG. 1A is a cross-sectional view along line 1A-1A of FIG. 1 illustrating the disposition of the jejunal and gastrostomy tubes and the connecting line.

FIG. 2 is a perspective partially cutaway view of an earlier feeding tube installed in a patient.

FIG. 3 is a perspective view of one embodiment of a catheter of the present invention.

FIG. 3A is an enlarged perspective view of an alternate embodiment of the distal portion of the catheter shown in FIG. 3.

FIG. 3B is a perspective view of alternate embodiment of the catheter shown in FIG. 3, wherein the catheter in FIG. 3B does not show a retention mechanism and does not have the optional distal tip.

FIG. 4 is an overhead view of the head of the catheter shown in FIG. 3.

FIG. 4A is a cross-sectional view of the catheter of FIG. 4, taken along line A-A.

FIG. 4B is a cross-sectional view of the catheter of FIG. 4, taken along line A-A, illustrating the presence of an optional guide wire.

FIG. 4C is a enlarged cross-sectional view along line 4B-4B of FIG. 3 illustrating the orientation of the lumens of the catheter in one embodiment.

FIG. 5 is a perspective view of an alternate embodiment of the head of a device of the present invention.

FIG. 5A is a perspective view of another embodiment of the head of a device of the present invention.

FIGS. 6 and 6A are perspectives view of the device of FIG. 3 showing the retention mechanism in an inflated or expanded position.

FIG. 7 is a cross-sectional view of one embodiment of an adapter suitable for use with the present invention.

FIG. 7A is a cross-sectional view of one embodiment of another adapter suitable for use with the present invention.

FIG. 8 is a perspective view of the abdominal wall trocar of the invention.

FIG. 9 is a side elevational view of the sharpened end of the trocar.

FIG. 10 is a perspective view of the tubular sheath for receiving the trocar of the invention.

FIG. 11 is a side elevational view of the beveled end of the sheath.

FIG. 12 is an end view of the beveled end of the sheath.

FIG. 13 is a partial side elevational view of the flanged end of the sheath.

FIG. 14 is an end view of the flanged end of the sheath.

FIG. 15 is a side sectional view of the sheath with the abdominal wall trocar telescopically received therein.

FIG. 16 is a perspective view of the gastrostomy introducer of the invention.

FIG. 16A is an end view of the tapering free end portion of the introducer.

FIG. 16B is a partial enlarged sectional view of the tapering free end portion of the introducer, as seen along line 16B in FIG. 16A.

FIG. 16C is an enlarged end view of the outer end of the introducer.

FIG. 17 is a perspective view of the elongated slotted cannulator of the invention.

FIG. 17A is a sectional view through the cannulator as taken along line 17A-17A in FIG. 17.

FIG. 18 is a side elevational view of the elongated slotted cannulator as taken along line 18-18 in FIG. 17.

FIG. 19 is a diagrammatic view showing the trocar and sheath combination inserted, inside to out, through the abdominal wall.

FIG. 20 is an enlarged diagrammatic view showing the feed tube inserted through the sheath in the abdominal wall after removal of the trocar.

FIG. 21 is a diagrammatic view showing the tubular gastrostomy introducer being inserted through the gastric wall into the lumen of the stomach.

FIG. 22 is a partial sectional end view showing the cannulator positioned over an inlet opening of the introducer, but spaced therefrom for operation as a sump.

FIG. 23 shows the introducer and cannulator operating in combination as a sump to aspirate the stomach.

FIG. 24 is a diagrammatic illustration showing the cannulator protruding through the pylorus after removal of the introducer.

FIG. 25 is a diagrammatic illustration showing the insertion of the feed tube through the cannulator beyond the pylorus and into the duodenum.

FIG. 26 is a diagrammatic illustration of the cannulator being withdrawn from the gastric wall with the feed tube being peeled from the longitudinal slot of the cannulator.

FIG. 27 is a diagrammatic illustration of the feed tube secured relative to the abdominal wall with the free end of the feed tube positioned in the jejunum beyond the Ligament of Treitz for effective nutritional support.

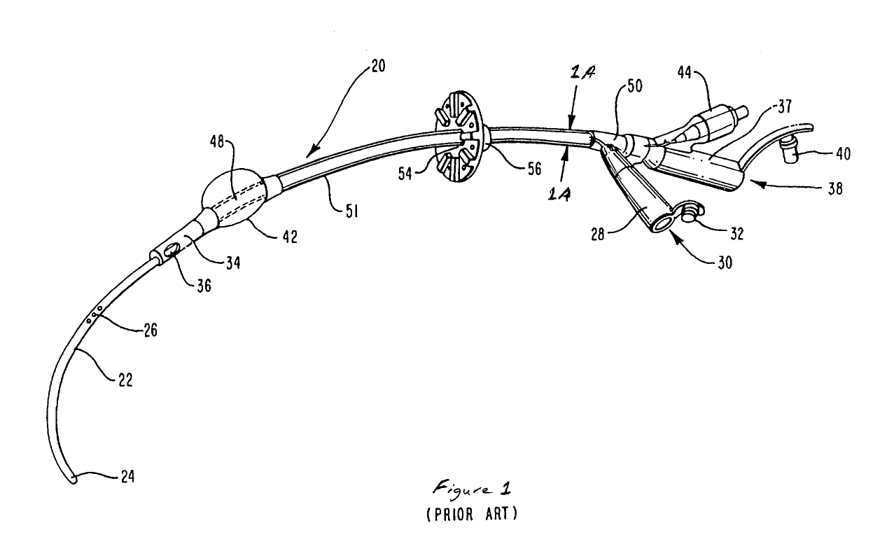

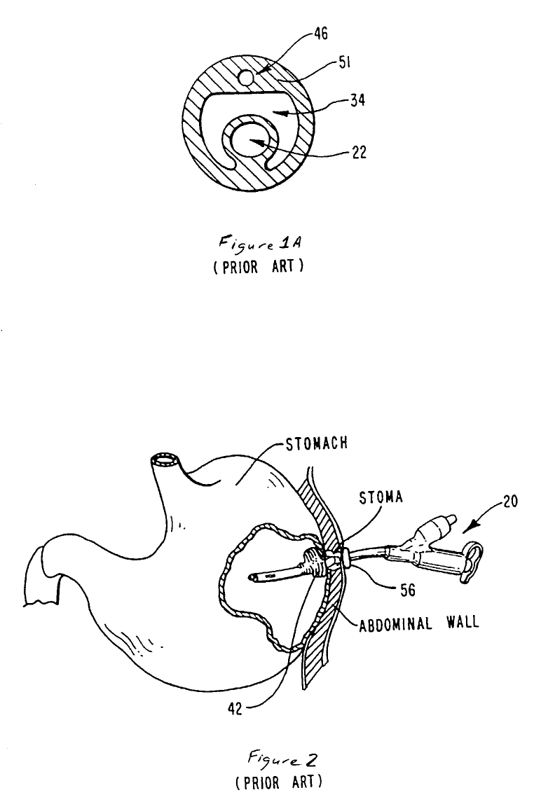

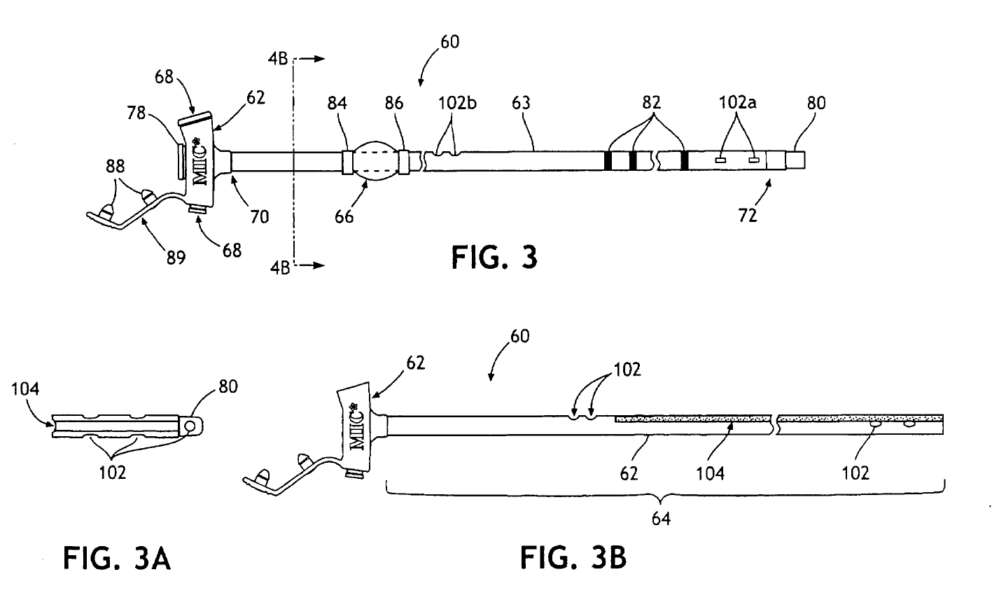

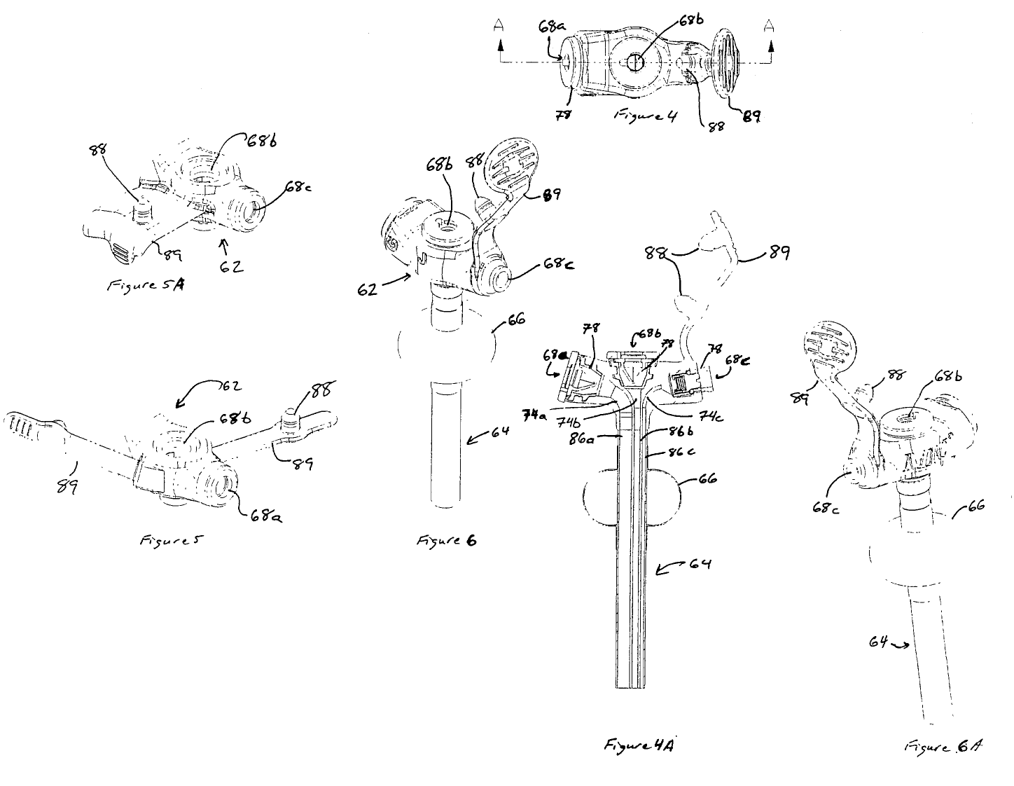

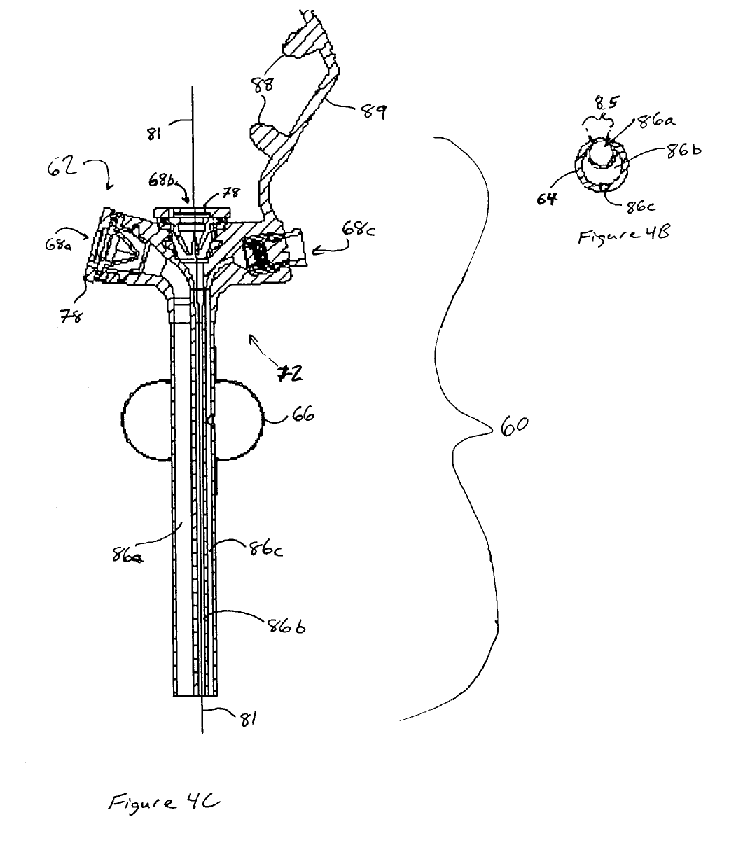

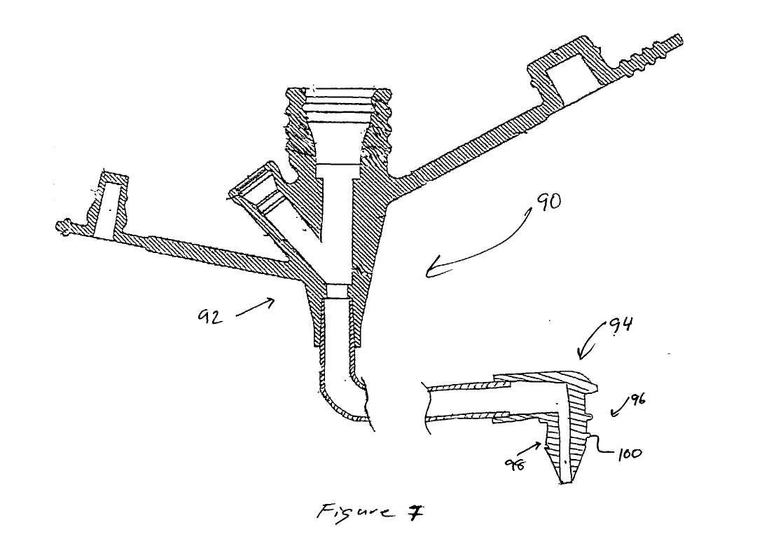

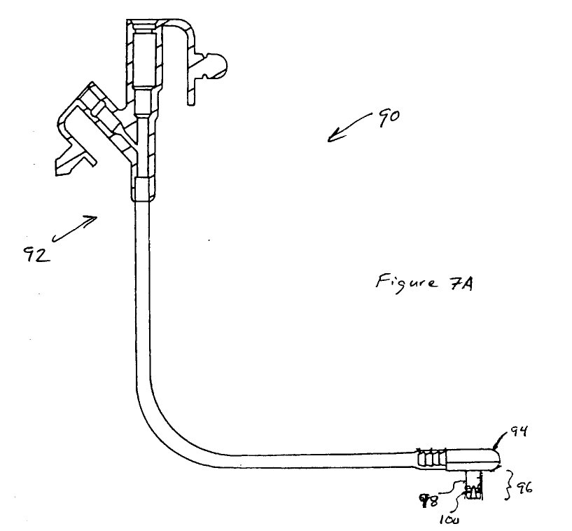

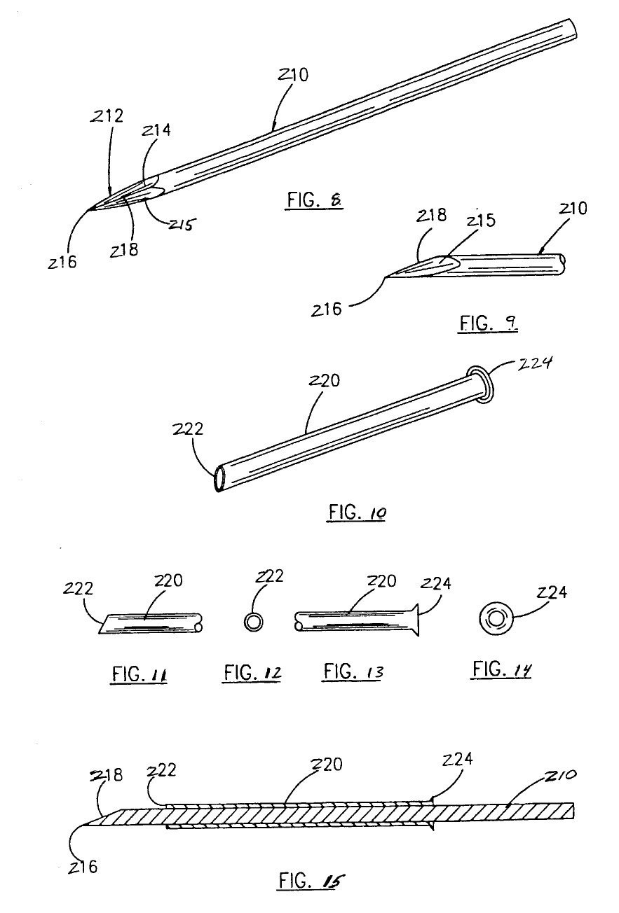

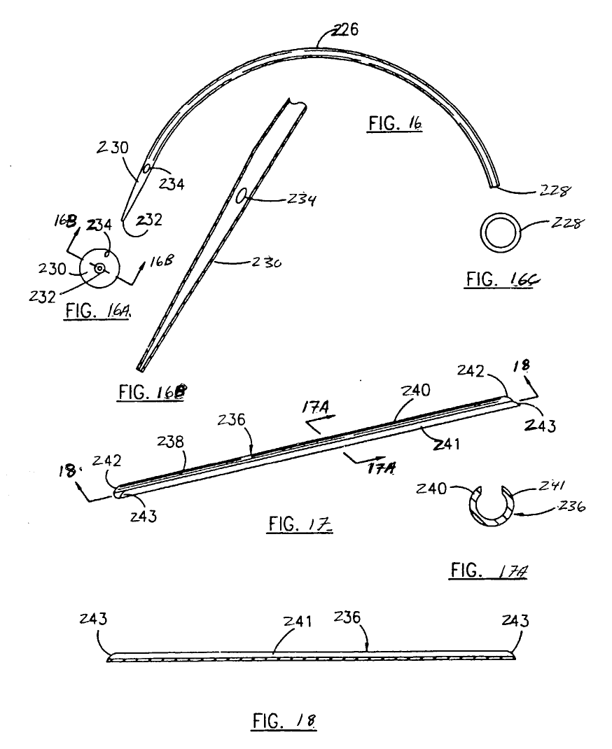

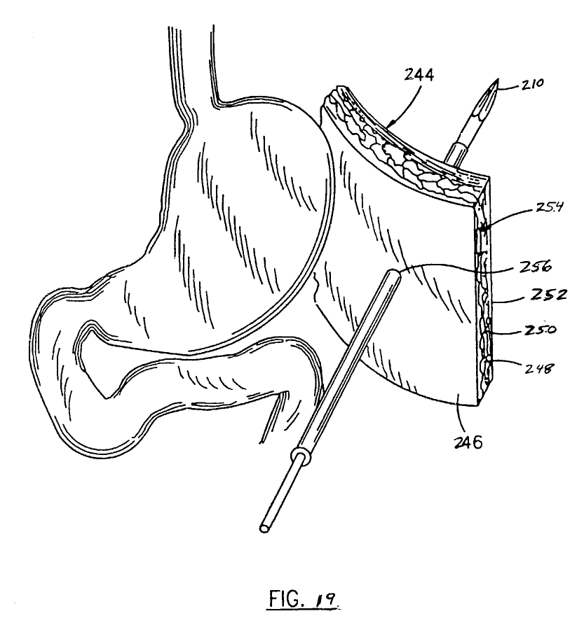

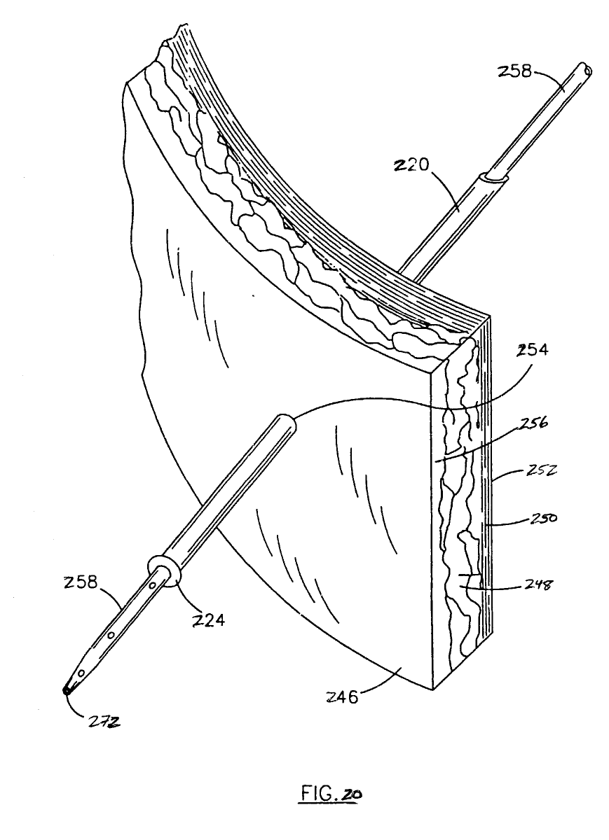

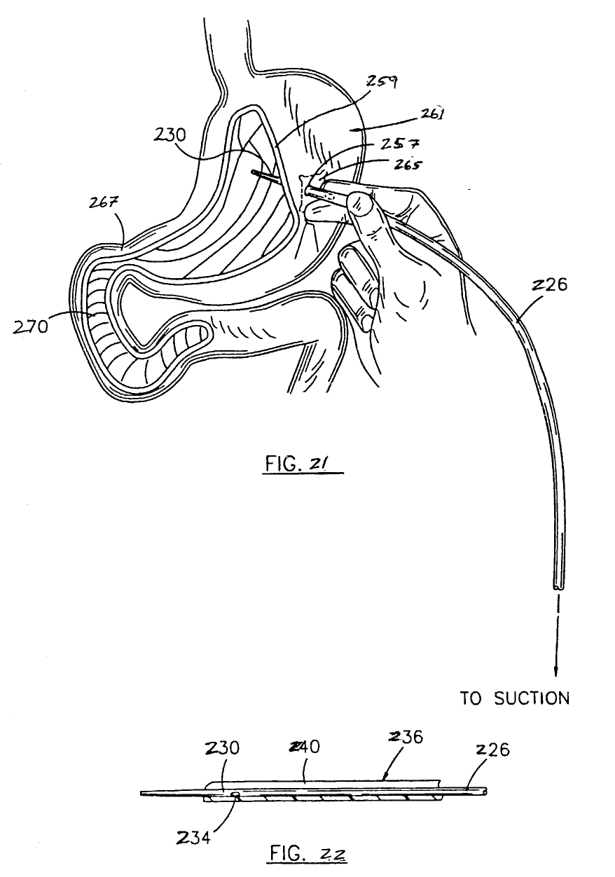

### DETAILED DESCRIPTION OF THE INVENTION

The following detailed description will be made in the context of a low profile catheter or transjejunal tube. It is readily apparent, however, that the article of the present invention would also be suitable for use as other types of catheter products and devices and the like. In addition, the invention will be described in the context of its various configurations. It should be appreciated that alternative arrangements of the invention can comprise any combination of such configurations. As such, the use of a preferred embodiment, a balloon catheter, for ease in understanding and describing the invention shall not, in any manner, limit the scope of the invention.

It is of note that the term “transjejunal” is frequently interchanged with the terms “transgastro-jejunal” and/or “gastro-jejunal”. While the term “transjejunal” is used throughout this disclosure, the term is intended to include the terms “transgastro-jejunal” and “gastro-jejunal” and/or any other term known to those having skill in the art which has a similar or an equivalent meaning.

The articles of present invention advantageously exhibit a low profile relative to the patient, and more preferably relative to the abdomen of the patient, when the article is properly positioned within a patient. The low profile attributes results in a reduction in the amount of the device which is positioned external to the patient as compared with a traditional or non-low profile device. The reduction in tubing and/or other portions generally found external to the patient in low profile devices as compared with non-low profile devices should provide for a reduction in the number of accidental displacements or removals of the articles. This will be true because the reduction in exposed tubing and/or size of the head attached thereto reduces the likelihood that a patient, especially a patient who is ambulatory or one who is active or prone to seizures, may somehow catch, snag, or entangle the protruding portion of the device in clothing, furniture, or the like thereby resulting in the unintentional or undesired dislodgement or removal of the device. Additionally, the low profile characteristics of the device may also provide improved or more preferable aesthetic and cosmetic characteristics for the device in that many patients, especially those who are ambulatory and who do not wish others to know about their condition or the presence of an enteral feeding device, will appreciate that the low profile head allows for a patient to more readily hide the protruding portion of the device below clothing.

Referring now to the FIGS. 3-6A, the present invention provides a catheter 60 having a head 62, a catheter segment 64, and a retention mechanism 66. The head of the catheter has at least two openings 68a and 68b through which fluid may pass to or from a patient, the head 62 having a low profile relative to a patient when the catheter 60 is properly positioned in the patient. As noted above the catheter of the present invention is preferably situated such that the head is positioned adjacent the abdomen of the patient, however, it may be preferable in some circumstances for the head 62 of the catheter to be positioned elsewhere on the patient. The catheter segment 64 extends from the head 62 of the catheter and has a proximal end 70 and a distal end 72. The catheter 60 also has at least a first lumen 86a and second lumen 86b, each of the lumens having a proximal end 74a, 74b and a distal end (not shown), and each lumen being in communication with at least one of the openings 68 of the head 62. The openings 68 in the head of the catheter are designed to allow for the passage of fluid into the catheter 60 and/or out of the patient. Depending on the embodiment of the invention, one or more of the openings 68 may also preferably include a valve 78 configured for selectively controlling fluid flow through one or more of the lumens of the catheter. Suitable valves 78 include, for example, one-way, two-way, and duckbill valves. The valves 78 can assist with the regulation of fluids to and/or from the patient. For instance, it may be preferable for one or more of the openings 68 in communication with the lumen or lumens which are used to provide nutrients, medicine and/or other fluids to the patient (as discussed below) to contain a one-way valve, such that the fluid or fluids intended to pass into the patient cannot or do not flow back out through the head of the catheter or so that fluids from within the patient are not unintentionally allowed to flow out of the catheter or at least certain lumens of the catheter. Alternately, an opening in the head 62 of the catheter 60 which is in communication with the stomach or gastric cavity of a patient may have a valve 78 which permits fluid to flow through the head 62 and into the patient as well as to permit flow of a fluid from the patient into the catheter 60 and out an opening 68 (e.g. venting), as discussed in more detail below. Further, where the retention mechanism is a balloon or elastomeric sleeve, as discussed below, it may be preferable for the opening 68 to include a one way check valve which can assist with inflation and/or deflation of the balloon as well as maintaining the preferred level of inflation.

Each of the lumens of the catheter 60 includes an entry port (not shown) in the proximal portion of the lumen and an exit port (not shown) in the distal portion of the lumen. More specifically depending on the formation of the head and lumens of the catheter, the entry port of the lumen may be one of the openings 68 in the head 62 of the catheter. Whether the lumens are integrally formed with the head of the catheter (as illustrated in FIG. 4A) or require a connection thereto, each lumen 86 in the catheter should be in communication with at least one opening 68 of the head 62.

Preferably, the distal end 72 of the catheter segment 64 may be adapted to terminate within the jejunum of a patient; however, the ultimate location or positioning of the catheter, including its distal end 72, will vary from patient to patient and will depend to some degree on the method of insertion or placement as well as the person who places the catheter in the patient. As such, in use the distal end 72 of the catheter segment 64 need not be placed, or it may be undesirable to place, the distal end such that it terminates within the jejunum of the patient, but the devices of the present invention have that capability. That is in use, although not the preferred use, the distal end of the devices of the present invention may not be placed or positioned so as to terminate in the jejunum, but rather may terminate in the small intestine of the patient. It is appreciated that people are of various sizes and weights and as such the location of certain body parts will not be the same from person to person. Accordingly, the present invention may be manufactured in a variety of sizes and lengths, such that patients of all sizes can be accommodated. For example, low volume balloons may be used on pediatric or pediatric sized catheters. All dimensions of the devices are envisioned to be variable and/or to have components that are scalable collectively or individually.

To assist with the placement of the devices of the present invention, the catheter segment 64 preferably includes a catheter shaft 63 having a distal end 72 and a stiff tip 80 attached to the distal end of the catheter shaft. In addition to providing a stiff tip 80 which can assist or enable penetration of certain portions of the patient's anatomy such as the Ligament of Treitz and the duodenum, the stiff tip 80 may also provide rigidity which can assist in the placement of the distal end 72. Furthermore, the stiff tip 80 can act to resist collapse or crushing of the end of the catheter, thereby preventing or reducing the likelihood of blockage or clogging of the catheter. The tip 80 is preferably rounded and has a soft tip to prevent or reduce abrasion. The catheter 60 may also include markers or markings 82 along the catheter segment 64, which may assist the person placing the device. The markings 82 may be visible without the assistance of other devices, (e.g. printed on the exterior of the catheter segment) or may require other means to view (e.g. radiopaque markers visible in x-rays) as discussed below.

In the present invention, a retention mechanism 66 is used to assist with the positioning of the catheter 60. Once activated or triggered, the retention mechanism 66 serves to keep the catheter in place such that it is not readily removed or dislodged from the patient. The retention mechanism 66 generally extends or expands in such a way that the catheter 60 cannot be readily withdrawn until the mechanism is withdrawn or deflated. When the mechanism is in its withdrawn or deflated state as shown in FIGS. 3, 25 and 26, the catheter is able to be readily moved in and out of the stoma through which it is inserted. The retention mechanism 66 is located along the catheter segment 64 at a position between the proximal end 70 and the distal end 72. Any location along the catheter segment 64 is suitable for the retention mechanism 66 provided it allows the desired retention to occur. Preferably, the retention mechanism 66 is positioned along the catheter segment 64 such that when engaged, triggered, inflated, or the like, the retention mechanism will be positioned adjacent to the abdominal wall of the patient. Alternatively, depending on the type of mechanism being used, it may be preferable for the retention mechanism to be positioned along the catheter segment such that when activated, the mechanism comes in contact with a duct within the patient in such a manner so as to create the desired retention function. Furthermore, when used within a duct within a patient, the retention mechanism 66, may also act to seal off the duct to prevent or reduce backflow through the duct. As one skilled in the art, any suitable retention mechanism may be used with the present invention, however, depending on the portion of the patient which the mechanism may contact, certain types of mechanisms may be more suitable than others. Several types of retention mechanisms are known to those skilled in the art. Suitable examples include, but are not limited to, those disclosed in U.S. Pat. No. 5,073,166 to Parks et al. and U.S. Pat. No. 6,264,631 to Willis et al.

While any suitable retention mechanism will work, it may be preferable to use a balloon or an elastomeric sleeve 66 as the retention mechanism. Preferably, such a balloon 66 may be formed by a sleeve with a first end attached to the exterior of the catheter segment 64 so as to form a first cuff 84 and a second end attached to the exterior of the catheter segment so as to form a second cuff 86, the balloon 66 collapsing on the catheter segment 64 when not inflated to thereby facilitate insertion or removal through a stoma. Although, it may be preferable for the sleeve or balloon 66 to be attached to the exterior of the catheter segment 64, it is contemplated that in one or more embodiments one or both ends of the sleeve or balloon may be secured to the interior of the catheter segment 64. Regardless of the type of retention mechanism used or the placement of the retention mechanism within the patient (e.g. against the stomach wall or in a duct), it is preferred that the retention mechanism hold the catheter in the place such that the low profile head generally rests against the body of the patient, and more preferably in a close or snug fit with or adjacent to the abdomen of the patient, but preferably not so close or snug so as to cause discomfort to the patient as a result of squeezing or pinching.

Another preferred characteristic of the catheter 60 of the present invention is that the low profile head 62 may have three openings 68a, 68b, and 68c (as shown in FIG. 4A) and the catheter segment 64 may have three lumens 86a, 86b, and 86c (as shown in FIG. 4A). Referring now to the catheter 60 shown in FIG. 4A, the low profile head 62 includes a first opening 68a for communication with a lumen 86a intended for fluid communication between the low profile head 62 and the jejunum of the patient; an opening 68b for communication with a lumen 86b intended for fluid communication between the low profile head 62 and a visceral organ of the patient; and an opening 68c for communication with a lumen 86c intended for communication between the low profile head 62 and the retention mechanism. While the catheter of FIG. 4A illustrates a low profile head 62 with three openings and a portion of catheter segment 64 having three lumens 86a, 86b, and 86c, the present invention contemplates and one skilled in the art will appreciate that additional openings and/or lumens may be included, limited only by the size, shape and intended use of the catheter.

In another embodiment, the low profile head 62 of the catheter 60 has a plurality of external surfaces and the openings 68 of the low profile head 62 may be on one or more surfaces of the low profile head. That is, depending on the shape and size of the head of the catheter, the openings in the head of the catheter may all be on one surface of head or the openings may be on multiple surfaces of the head. One skilled in the art will appreciate that the size and shape of the low profile head may vary, and that additional components (e.g. additional openings, etc.) may be included in those devices having larger heads. All variations in size and shape of the head of the catheter as well as the size, spacing and orientation of the openings around the head are contemplated by the present invention and are intended to be covered by and included in the claims of the present invention.

The present invention may further include a protective covering 88 (as shown in FIGS. 3, 3B, 4, 4A and 5-6A) for at least one of the openings 68. Any suitable protective covering 88 is acceptable. Examples of suitable protective coverings 88 include, but are not limited to, plugs, caps, inserts and the like. Some embodiments may include a protective covering which is attached to or formed as a part of a strap 89. Such a strap 89 may have one or more protective coverings 88 attached thereto or formed thereon depending on the design of the catheter head and the orientation of the openings in the head (see, for example, FIGS. 3 and 5A). Other embodiments may have multiple straps (see, for example, FIG. 5) or may not have any straps or protective coverings at all (not shown). Where multiple protective coverings 88 are included on one strap 89, the coverings will preferably be designed such that they may fit any of the openings 68 they may be inserted into. That is, it is preferable for the openings and/or the protective coverings to be universal in size and shape such that the coverings are interchangeable among the openings, limited only by the ability of a covering 88 to reach an opening 68 because of the length of the strap, cord or the like 89 to which the covering 88 is attached. It will be appreciated that the strap, cord or any other suitable attachment mechanism 89 may be formed integrally with a portion of the device of the present invention as shown, for example, in FIGS. 3 and 5, or the suitable attachment mechanism 89 may be separately secured or attached to the device of the present invention. That is, in one embodiment the proximal end of the catheter segment may include at least one external retention member or attachment mechanism 89 and a protective covering 88 formed with the external retention member 89. Further where the attachment mechanism or external retention member 89 is not formed integrally with the present invention, the external retention member may include a means for securing (not shown) the protective covering to the catheter. It is appreciated that the means for securing the protective covering may 88 or may not be integrally formed with mechanism or member 89.

As noted above, the head 62 may be of different sizes and shapes. Additionally, depending on the size of the catheter 60 being used, one skilled in the art will recognize that the head 62 of the catheter 60 may have a larger cross-sectional diameter than the catheter segment 64. Further still, the head of the catheter of the present invention may be capable of receiving an adapter 90 (as shown in FIGS. 7 and 7A). That is, in at least one embodiment of the present invention, it may be preferable for at least one of the openings 68 of the low profile head 62 to be capable of receiving an adapter 90. The present invention may further include an adapter for communication with one or more of the lumens of the catheter with the adapter having a proximal end 92 and a distal end 94 and one or more lumens which allow fluid communication between the proximal end 92 and the distal end 94 of the adapter 90. Suitable exemplary adapters include, but are not limited to, those described and disclosed in U.S. Pat. Nos. 5,073,166 and 6,264,631 and U.S. patent application Ser. No. 09/660,665, each of which is incorporated by reference in their entirety, as well as those known to those skilled in the art, such as feeding tubes, infusions sets, and the like. Those embodiments capable of receiving an adapter preferably have a positioning mechanism (not shown) capable of enabling proper alignment of the lumens in the catheter with the one or more lumens in the adapter. That is in those embodiments where the one or more openings in the head of the catheter is in communication with more than one lumen, the catheter may contain a means for enabling or ensuring that the lumens in the adapter are properly aligned with the lumens in communication with the opening to which the adapter is connected such that fluids intended to pass through one lumen of the catheter do so and are not inadvertently or unintentionally allowed to pass through one or more of the other lumens of the catheter.

Some of the advantages of the low profile aspects of the present invention are discussed above, however, the use of an adapter, extension sets or the like 90 may provide additional advantages. For example, in non-low profile devices, it is common for the portion of the device or apparatus which extends from the patient (e.g. that portion of the tube or catheter external of the patient's abdomen) to become clogged. While the reasoning is not known with specificity, it is has been observed that the portion of a feeding tube or catheter which extends beyond the patient has a greater incidence of clogging than the portion of the tube or catheter which is inside the patient. As such the need to replace the device comes about more frequently because of clogging external to the body than would be necessary because of clogging experienced in the portion of the tube or catheter inside the patient. As such the present invention will reduce the need for or extend the time between the replacement of the tube or catheter as the adapter(s), extension sets, and the like 90 which connect thereto may be readily removed, cleaned and replaced or replaced all together with a different adapter, extension set or the like may be removed and cleaned and then replaced. The reduction in the number of times the catheter must be replaced is highly preferred not only because of the reduction in the procedures, but also, for example, because of the reduction in exposure to trauma which the patient may experience because of the increased number of procedures.

Preferably the catheter 60 may also include a means for releasably latching the adapter 90 to the low profile head 62 of the catheter. Any suitable means for releasably latching the adapter will work and will be appreciated by those skilled in the art. As shown in FIG. 7A, an exemplary latching means 96 may include a leg 98 extending from the distal end 94 of the adapter 90 and a finger 100 formed at a free end of the leg 98 and extending at an angle generally perpendicular relative to the leg 98. The leg 98 and finger 100 serve to guide the adapter 90 into the latching means 96. Once inserted into the head of the catheter, the distal end 94 of the adapter 90 may, for example, be rotated such that the adapter 90 is retained in place until rotated back to the insertion position. In embodiments of the present invention where the adapter 90 contains multiple lumens the latching mechanism or means 96 may also serve or function to ensure proper alignment of the lumens of the adapter with the appropriate lumen or lumens of the catheter.

One skilled in the art will appreciate that each of the lumens 86a, 86b, 86c of the catheter 60 preferably has at least one opening or aperture (not shown) at or near the distal end of the lumen. It will be appreciated by those skilled in the art that where a lumen extends beyond the opening or aperture at or proximate the distal end of the lumen, but where the lumen is plugged, filled or blocked (e.g. where a radiopaque material fills the distal end of the lumen, as discussed below), the point at which the lumen is plugged, filled or blocked shall be considered the distal end of the lumen. (This is best illustrated in FIG. 3B, where apertures 102b are near the end of the portion of the gastric or venting lumen (not shown) through which fluid may pass as, in that embodiment, the radiopaque material 104 fills the portion of that lumen in the distal portion of the catheter so as to create a plug which blocks the flow of fluid through that lumen beyond the point at which the radiopaque material 104 is positioned). The apertures 102 at or near the distal end (not shown) of the lumens provide for the flow of fluids therethrough to or from the exterior of the catheter. It will also be appreciated that preferred catheter segments 64 will have at least one opening 102 (as shown in FIG. 3B) therein corresponding to the one or more of the openings or apertures at or near the distal end of the lumens. The opening or aperture 102 in the catheter segment 64 which corresponds with openings in the distal end (not shown) of the lumens 86 allows fluid to pass therethrough so as to complete the communication between the head 62 of the catheter 60 and the exterior of the catheter segment 64. For example, nutritional fluids may preferably pass through the head of the catheter, through one of the lumens and out of the distal end of the lumen into the desired location in the patient; or gas may, for example, be vented by passing from the patient into one of the lumens and out through the head of the catheter. For purposes of this disclosure, fluids such as saline, which are provided to trigger the retention mechanism 66 (e.g. inflate or expand the balloon or sleeve) shall also be considered to pass to the exterior of the catheter segment 64 as those fluids pass through the head 62 of the catheter 60 into the lumen and outside the catheter segment 64 as the mechanism 66 expands even though the fluid may never pass to or from the patient through the lumen in communication with the retention mechanism 66.

It will also be appreciated that multiple apertures or openings may be present in catheter segment 64 and/or the distal end of the lumens 86 such that fluid flow is not inhibited should one or more of the other apertures 102 in a lumen or the catheter segment (for example, as shown in region 85 of FIG. 4B, depending on the formation of the catheter, the wall of the catheter segment 64 may be both a wall of a lumen and a wall of the catheter segment such that a opening created in one is an opening created in the other) become clogged or blocked. Further still, it will be appreciated by those skilled in the art that the size, shape and spacing of the apertures in the lumens or catheter segment may vary and will depend in part on the desired application. All such variations are contemplated and intended to be included and covered by the present invention. For example, the number and/or size and/or location of apertures along a lumen or catheter segment may vary depending on the size (both diameter and length) of the catheter segment and lumens being used. That is, for example, the number and size of the apertures in the catheter segment in an adult catheter may not be the same as a pediatric catheter or even an adult catheter having different length catheter segments.

Depending on whether the catheter is to be placed during a surgery or a less traumatic fashion or to be used as a replacement for a pre-existing device, it may be preferable for the catheter to also include or be used in connection with a guide wire 81. During use or placement the wire may extend through the catheter, preferably through the head and a opening at the distal end 72 of the catheter (as shown in FIG. 4C). The catheter may be slid over the guide wire 81 to assist with the placement of the catheter, or in other embodiments the wire may be used to control or steer the distal end or distal tip (not shown in FIG. 4C) of the catheter 60 to better enable preferred placement of the device.

Yet other embodiments may comprise additional features or components, such as a radiopaque material or marker 104. The radiopaque material 104 may be positioned at one or more points along the catheter 60, but is preferably positioned at or near the distal portion of the catheter segment 64, such that the location of the distal end 72 of the catheter, and more particularly the distal tip 80, if present, may be verified by x-ray or other suitable means. The radiopaque material 104 may be used in a variety of forms, such as tubes, plugs, stripes, markers, tips, and the like with the preferred form depending on the application and the person placing the catheter. In some embodiments, it may be preferable to fill or substantially fill a portion of a lumen with a radiopaque material. Such an embodiment is shown in FIGS. 3A and 3B where the distal portion of the lumen having apertures is filled with tungsten (or a tungsten containing material or powder) 104, such that the tungsten provides the ability to determine the position of the portion of the catheter in which the tungsten is present without inhibiting or hindering the operational ability of the remainder of the catheter and more specifically without interfering with the apertures 102 along the catheter segment. One skilled in the art will recognize that there are a number of suitable radiopaque materials and that any suitable material may be used in the present invention. As noted above, an example of such a suitable material is tungsten or a tungsten containing powder or material.

The present invention is also directed to a balloon catheter adapted for placement through a stoma into a body cavity so that the balloon catheter is maintained in the stoma. The balloon catheter includes a head having at least two openings through which a fluid may be passed, the head being low profile relative to a patient, and preferably the patient's abdomen, when the catheter is correctly positioned within the patient; a catheter segment extending from the head to a distal tip, the catheter segment having an exterior and a wall defining at least two passageways through the interior; and an elongate sleeve attached to the exterior of the catheter segment about the passageway so as to form an inflatable balloon which covers a portion of the catheter segment. The sleeve is preferably formed such that in its collapsed state (i.e. when not inflated) the sleeve closely surrounds the catheter segment, and when inflated the sleeve extends radially outwardly from the catheter segment. In this embodiment, the inflation of the sleeve may be controlled through one of the openings in the head of catheter as the sleeve is in communication with one of the openings in the head of the balloon catheter. Preferably the passageway through the interior of the catheter segment has at least a plurality of lumens, at least one of which extends from the head of the catheter to a point at or near the distal end of the segment, and at least one of which extends from the head of the catheter to the elongate sleeve.

A preferred embodiment of the balloon catheter may also have one or more of the following attributes: the attachment of the elongate sleeve to the exterior of the catheter segment forms a proximal cuff and a distal cuff of the balloon; the catheter segment includes a catheter shaft and a stiff tip attached to a distal end of the catheter shaft, opposite the head; one of the lumens of the catheter is a feeding tube, the distal end thereof which is preferably adapted to terminate within a jejunum of a patient; one of the lumens may be a venting lumen, wherein fluid contained within a visceral organ of a patient may be evacuated through the venting lumen; and one or more of the openings of the head of the catheter may be capable of receiving an adapter. The ability to receive an adapter provides the patient the ability to temporarily attach or to allow connection to a feeding tube, infusion set or the like at desired times, while providing a more compact apparatus when not attached thereto, when a larger more conspicuous apparatus may be undesirable. For example, a larger more conspicuous apparatus or device may be undesirable when in public or if a patient is ambulatory or affected by seizures such that a longer extension is more likely to get caught on or become entangled in something, possibly resulting in the unintentional removal or partial removal of the device. As discussed in more detail below, the ability to use an adapter or the like also reduces the frequency at which the catheter needs to be replaced, as the adapters or other connections may be removed and cleaned or discarded and replaced.

Yet another embodiment of the present invention defines a balloon catheter adapted for placement through a stoma into a body cavity so that the balloon catheter is maintained in the stoma. The balloon catheter includes a low profile head having at least three openings through which a fluid may be injected, the head being low profile relative to a patient's body when the catheter is correctly positioned; a catheter segment extending from the head portion to a distal tip, the catheter segment having an exterior and a wall defining a passageway through the interior, the passageway defining at least three lumens, each lumen having a proximal end and a distal end and each extending from the head of the catheter at least a portion of the length from the head of the catheter to the distal end of the catheter; and an elongate sleeve attached to the catheter segment about the passageway so as to form an inflatable balloon which covers a portion of the exterior of the catheter segment, the elongate sleeve being in communication with one of the at least three lumens, the sleeve being in a collapsed state when not inflated wherein the sleeve closely surrounds the catheter segment, and wherein the sleeve extends radially outwardly from the catheter segment when inflated. Preferably, each of the at three lumens has one or more opening near the distal end of the lumen which provides for communication exterior of the catheter. This communication may, for example, be the excretion of fluids being passed through the head and lumens of the catheter which are intended to provide nutrition or medicine to a patient, the removal of fluids or gases from the patient through the catheter or the inflation and deflation of the retention mechanism which is secured to the catheter segment. Again, it is preferable that the attachment of the elongate sleeve to the exterior of the catheter segment occurs so as to form a proximal cuff and a distal cuff of the balloon. Further still, the catheter segment preferably further includes a stiff tip attached to the distal end of the catheter segment (opposite the head) and/or the catheter segment may also include an annular recess, wherein the sleeve is disposed in the annular recess when it is in a deflated state.

Another embodiment of the present invention defines a catheter including a head, the head comprising at least three openings through which fluid can be introduced, the head having a low profile; a catheter segment extending from the head, the catheter segment having a proximal end and a distal end, the catheter segment further including at least a first lumen, a second lumen, and a third lumen, each of the lumens having a proximal end and a distal end, and each lumen being in communication with at least one of the openings of the head; and a retention mechanism attached to the catheter segment at a position between the proximal end and the distal end of the segment. Preferably, the head of the catheter is designed such that the first one of the three openings in the head is adapted for communication with a first one of the three lumens in the catheter segment such that fluid communication may be achieved between the head and the jejunum of a patient; that a second one of the three openings in the head is adapted for communication with a second of the three lumens in the catheter segment such that fluid communication may be achieved between the head and a visceral organ of the patient; and a third one of the three openings in the head is adapted for communication with a third of the three lumens in the catheter segment such that communication may be achieved between the head and the retention mechanism. It is contemplated that the communication between the third opening in the head of the catheter and the retention mechanism may include, without limitation, fluid communication or triggering communication, depending on the type of retention mechanism used. That is, for example, where a balloon or elongate sleeve is retention mechanism the third opening in the head of catheter will preferably be adapted for fluid communication with the third lumen of the catheter. By way of further example, where the retention mechanism is triggerable (as discussed above) rather than inflatable, the third opening in the head need only be in or allow communication with the third lumen of the catheter (and ultimately the retention mechanism) which is sufficient to allow the retention mechanism to be triggered, released, activated or the like as well as to allow for retraction, deactivation or the like thereof. As way of an example of a suitable communication where a non-balloon or elongate sleeve retention mechanism is used, the third opening in the head of the catheter may be capable of accommodating a stylus which may pass through the opening and through or into the third lumen of the catheter so as to enable an operator to trigger or otherwise activate to enable the retention mechanism and/or to release or deactivate the retention mechanism to enable withdrawal thereof. As noted above the retention mechanism preferably may be a balloon and the balloon may be formed by a sleeve with a first end attached to the catheter segment so as to form a first cuff and a second end attached to the catheter segment so as to form a second cuff, the balloon collapsing on the catheter segment when not inflated to thereby facilitate insertion or removal.

The catheter segment of the present invention may also include a catheter shaft having a distal end and a stiff tip attached to the distal end of the catheter shaft. Preferably one or more of the openings in the head has a valve configured for selectively controlling fluid flow through one or more of the lumens. To reduce contamination of the catheter or of the patient the catheter may also include a protective covering for at least one of the openings. Any suitable protective covering may be used. Suitable protective coverings may include, for example, a cap, plug, or the like (as discussed in more detail above). Preferably the protective covering will also include a strap with at least one plug or the like for insertion into one or more of the openings. In a preferred embodiment, the proximal end of the catheter segment may include at least one external retention member or securing mechanism and a protective covering formed with the external retention member or securing mechanism, and wherein the external retention member includes a means for securing the protective covering to the catheter.

Further, as noted above with other embodiments, at least one of the openings of the head of the catheter may be capable of receiving an adapter. The adapter is preferably adapted for communication with one or more of the lumens of the catheter, wherein the adapter has a proximal end and a distal end and one or more lumens which allow fluid communication between the proximal end and the distal end of the adapter. In at least one embodiment of the present invention, at least one of the openings of the head of the catheter has a positioning mechanism capable of enabling proper alignment of the lumens in the catheter with one or more lumens in the adapter.

Additionally, in those embodiments having adapters or capable of receiving adapters, it is preferable for the catheter to have a means for releasably latching the adapter to the head of the catheter. Any suitable means for releasably latching or otherwise attaching an adapter to the head of the catheter is contemplated by the means for releasably latching. An exemplary means for releasably latching an adapter to the head of the catheter may include a leg extending from the distal end of the adapter, wherein the means further includes a finger formed at a free end of the leg and extending at a generally perpendicular angle relative to the leg.

Lastly, one or more embodiments of the catheter of the present invention may include a catheter segment having a radiopaque material (as discussed in more detail above), the material being positioned at one or more points along the catheter segment and/or at least one opening which corresponds to one or more of the openings at or near the distal end of the lumens, wherein fluid may be allowed to pass therethrough.

The present invention is also directed to a transpyloric jejunostomy cannulating apparatus and includes several distinct devices including an abdominal wall trocar 210 shown in FIGS. 8 and 9. The trocar 210 is illustrated as an elongated length of a rigid rod, round in cross section and having a sharpened end 212. The sharpened end may be formed by a pair of angled machined cutting faces 214 and 215 which result in the formation of an extremely sharp tip 216 and cutting edge 218. The sharpened end 212 of the trocar may be alternately shaped so long as it provides an extremely sharp tip and cutting edge for passage through the abdominal wall. It is believed that passage utilizing a sharp trocar or the like will lead to a decreased rate of gastro-cutaneous fistula. The trocar may be made of aluminum, stainless steel or any other suitable material.

FIG. 10 illustrates the tubular sheath 220 which is adapted to receive the trocar 210 for telescopic sliding movement therein as illustrated in FIG. 15. The sheath 220 is of a size and shape to closely conform to the trocar for passage through an abdominal wall with it. As shown in FIGS. 8 and 9, one end 220 of the sheath is preferably beveled or cut at an angle to facilitate passage through the abdominal wall with the trocar.

FIG. 12 illustrates the round tubular cross section of the sheath and FIGS. 13 and 14 illustrate the opposite flanged end 224 of the sheath. The length of the sheath is not critical to the invention and a preferred length is approximately 20 centimeters. The sheath is preferably made of a semi-rigid plastic material.

FIG. 16 illustrates the elongated tubular gastrostomy introducer 226 which has one end 228 adapted for connection to a suction source and an opposite free end portion 230 tapering toward a free end 232 of reduced width for insertion through the gastric wall of a patient into the lumen of the stomach. The free end is preferably open as illustrated in FIGS. 16A and 16B and an additional larger inlet opening 234 is provided on the tapering free end portion 230 for use as a sump for aspirating the stomach.

FIGS. 17, 17A (taken along line 17A-17A of FIG. 17) and 18 (taken along line 18-18 of FIG. 17), illustrate the elongated slotted cannulator 236 of the invention. Cannulator 236 is generally C-shaped in transverse cross section as illustrated in FIG. 17A. It has an external width for passage through the sheath 220 and preferably has an internal width to accommodate passage of the introducer 226 through it. Preferably, the cannulator is of a size and shape to closely conform to the introducer 226 for passage through the gastric wall much like the sheath 220 conforms to the trocar 210 for passage together through the abdominal wall. The cannulator is preferably made of a semi-rigid plastic material such as polyethylene, but could be made of various suitable materials. It is designed to be disposable and somewhat flexible. The cannulator 236 should have sufficient column strength for passage of one end through the pylorus of a patient, as described hereinbelow, upon manipulation of the opposite (proximal) end from a position outside or external the gastric wall. Alternatively the cannulator 236 should have sufficient column strength to assist or enable passage of at least the distal end of a feed tube or catheter 258 through the pylorus of the patient even where the cannulator 236 does not extend into or pass through the pylorus. Note that the cannulator 236 has a continuous longitudinal slot 238 of a circumferential expanse sufficient to enable the feed tube or catheter 258, described below, to be peeled from the cannulator through the slot. The circumferential expanse of the slot 238 is generally between 45° and 90° and preferably between 70° and 80°. To facilitate passage of the cannulator 236 through the gastric wall (desirably with the introducer 226) the opposite side walls 240 and 241 of the cannulator 236 may have rounded tapered ends as indicated at 242 and 243. The length of the cannulator 236 is preferably at least 25 centimeters and a more preferred length is approximately 32 centimeters.

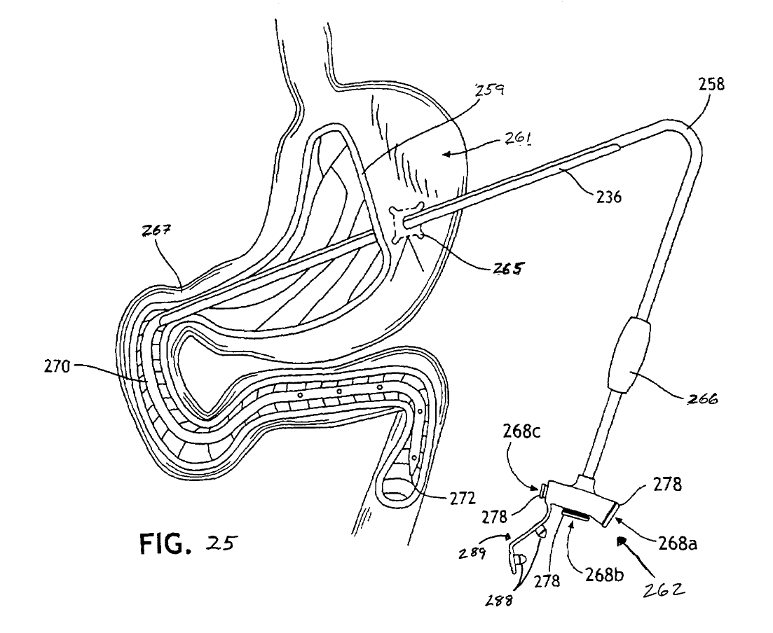

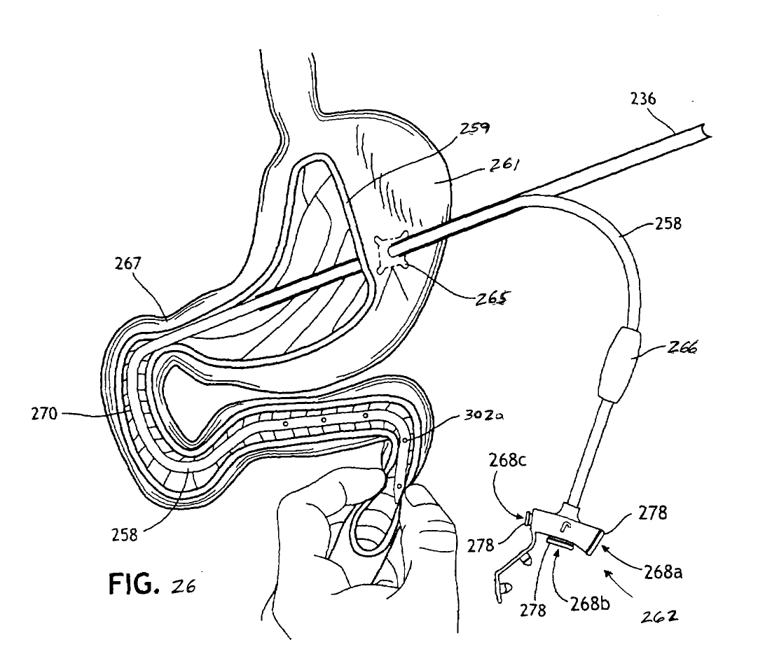

The elongate flexible tube 258 included in the apparatus discussed above has a proximal and a distal end, and has a low profile head 262 attached to the proximal end thereof. The tube 258 should preferably be of a length and width for insertion within or through the cannulator into the jejunum and beyond the Ligament of Treitz of a patient. The head 262 of the catheter 258 will have at least a plurality of ports 268a, 268b, and 268c. The flexible tube or catheter 258 preferably has at least a first, a second, and a third lumen defined within the tube. At least one of the lumens has an opening at the distal end of the elongated flexible feeding tube and is preferably adapted or configured to receive a guide wire (not shown). The apparatus, and more specifically, the flexible tube may further include a radiopaque material (shown in FIGS. 3A and 3B above as 104). At least one port 268 of the low profile head 262 should include comprises a valve 278. The low profile head 262 of the tube may be capable of receiving one or more adapters having multiple lumens therein. As noted above exemplary adapters are shown in FIGS. 7 and 7A. The tube may further include a means for releasably latching an adapter to the head of the apparatus. Additionally, as shown in FIG. 25, the tube 258 may further include an external retention member 289 and at least one protective covering 288 attached to the external retention member. The protective covering may be attached or secured to the external retention member in any number of suitable ways, including, but not limited to integral formation therewith. The apparatus may further include a means for securing the protective covering to the head 262. It is contemplated that any of the variations of tubes or catheters discussed above in more detailed may be used with the cannulating apparatus of the present invention. Additionally, the apparatus may further include an elongated generally tubular gastrostomy introducer 226 (shown in FIGS. 21-23) which has one end adapted for connection to a vacuum or suction source and a free end portion 230 tapering toward a free end 232 of reduced width adapted for insertion through the gastric wall into the lumen of a stomach. The introducer 226 may also have an inlet opening 234 adjacent the free end. Preferably, the elongated slotted cannulator 236, discussed in more detail above, will have an internal width to accommodate passage of the introducer 226 therethrough. Further still, it is preferable for the cannulator 236 to be of a size and shape to closely conform to the introducer 226 for passage through a gastric wall therewith.

The method of using the transpyloric jejunostomy cannulating system of the invention is illustrated in FIGS. 19 through 27. FIG. 19 shows the sheath 220 telescopically fit onto the trocar 210 with both being thrusted or inserted through the abdominal wall by an inside-to-out stab wound. It is preferred for this procedure to be performed during a surgical procedure such that the inside-to-out stabbing with the trocar may occur as shown; however, it is also possible for the trocar to be used in an out-to-inside stabbing action.

Solely for ease in understanding this method, the preferred procedure, which begins with an inside-to-out puncture of the patients abdominal wall with the trocar 210, shall serve as the groundwork for the remainder of the disclosure related to this method. Differences in the preferred procedure as compared with an outside-to-inside procedure will be noted where applicable. For example, one skilled in the art will appreciate that where the trocar 210 is used in an out-to-inside manner that the remainder of the procedure will be substantially the same as described below, except that the sheath 220 will also penetrate or pass through the tissues of the abdominal wall in an outside-to inside fashion and that references to the actions of a surgeon within the patients body may be modified so as to be accomplished from outside the patient or in some instances the action may be omitted.

The extremely sharp tip and cutting edge of the trocar 210 shown in FIG. 19 enables passage through the tissues of the abdominal wall including the peritoneum 246, abdominal wall muscles and facia 248, subcutaneous fat 250 and skin 252. The trocar 210 is preferably inserted through the abdominal wall at an oblique angle so that the passage through the abdominal wall is longer than the thickness of the abdominal wall. The formation of a long submuscular and subcutaneous tunnel by the trocar 210 allows optimum sitting of the peritoneal puncture wound 254 as well as the exit site wound through the skin. Optimum sitting of the peritoneal puncture should allow for good approximation of the fundus of the stomach to the peritoneum 246. Optimal sitting of the skin exit sites should allow the exit to be well away from the incision 256 and well away from other stomas or drains that the surgeon might employ. It is believed that the long oblique tunnel is further advantageous for decreasing the incidence of gastro-cutaneous fistula.

After puncturing the abdominal wall 244 the trocar 210 is withdrawn and a feed tube or catheter 258, described in further detail herein, may be inserted through the sheath 220. Sheath 220 may then be withdrawn.

It is contemplated that it may or may not be preferred to insert the feed tube through the sheath at this point in the procedure. As such, the tube may be inserted through the sheath later in the procedure, but preferably prior to the step of inserting the feeding tube 258 into the cannulator 236 and prior to advancing the distal end of the tube 258 into the jejunum beyond the Ligament of Treitz. The sheath 220 is also preferably withdrawn prior to the advancement or placement of the distal end of the tube or catheter 258 into the jejunum of the patient; however, as discussed below, a sheath which is capable of being peeled, split, or otherwise removed from around the catheter 258 can allow for later removal of the sheath 220.

Since the flanged end 224 of the sheath 220 is on the interior of the patient (as shown in FIGS. 19 and 20) during the preferred procedure of this type, it does not interfere with withdrawal of the sheath 220 through the abdominal wall. One skilled in the art will recognize that when a sheath 220 is used during an out-to-inside procedure (referred to above), the sheath 220 should be capable of being peeled away or otherwise removed away from the shaft of the tube or catheter 258 such that the removal or extraction of the sheath 220 is not blocked or otherwise prevented by the head 262 of the tube 258. Any suitable removal or separation technique may be used with the sheath. Other suitable removal or separation techniques include, for example, peeling away or splitting or slicing the sheath and then pulling it away or otherwise removing it from the tube.

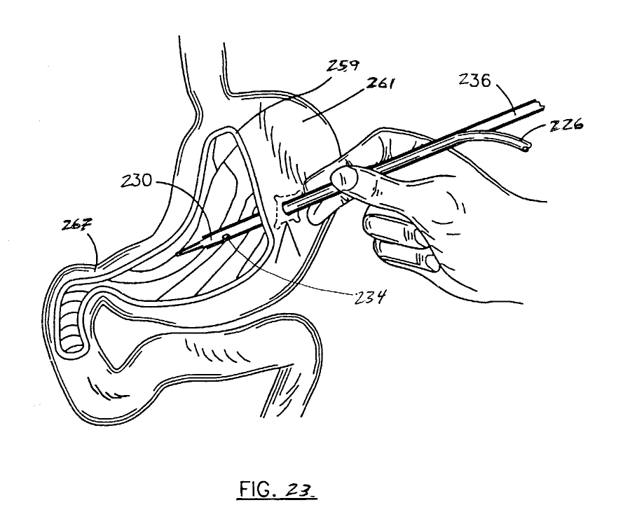

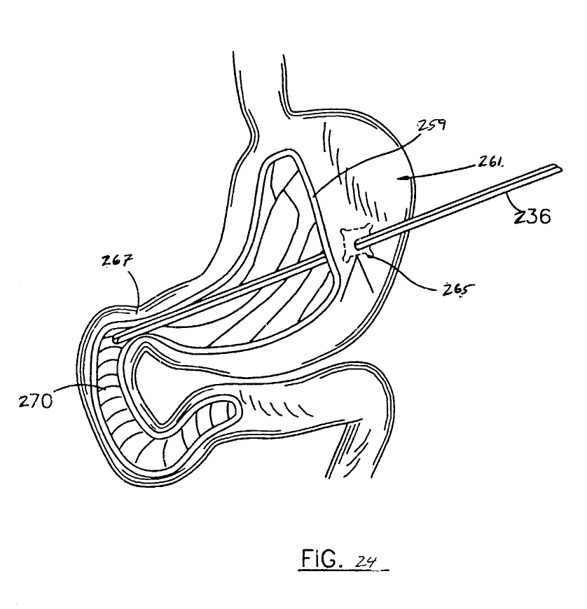

Next, a puncture site on the gastric wall 259 of stomach 261 should be located at a position for registration with the peritoneal puncture formed by the trocar 210. Once the preferred site is located the gastric wall should be punctured. The gastric wall hole 257 is preferably made by a scalpel or electro-cautery device to thereby form a small opening or hole. If the hole or opening 257 is not of sufficient or desired diameter, an introducer or dilator 226 may be used to increase the size of the opening so as to allow introduction of the cannulator. Preferably, the introducer 226 will have a free end 232 which has a diameter of only about 2 millimeters. One skilled in the art will appreciate that the size of the free end 232 of the introducer 226 as well as the size of the introducer 226 will depend on the size of the hole or opening 257 into which it is being inserted as well as the preferred size of the hole or opening 257 after insertion thereof. In use, the free end 232 of the introducer 226 may be inserted into the hole or opening 257. As the introducer 226 is pushed through the hole 257, the expanding width of the tapered free end 230 dilates the hole 257 to form somewhat of a seal around the introducer to prevent leakage of stomach fluids. Furthermore, a purse string suture 265 (shown in FIG. 21) around the gastric wall hole 257 may be used to further contribute to the sealed relation between the introducer 226 and gastric wall hole 257. Whereas the tapered free end 230 of introducer 226 is flexible, it is stiff enough to push through the hole 257 while, at the same time, being soft enough that, when pushed through the gastric wall, it will not pierce the opposite side of the stomach. Upon initial insertion of the introducer 226 through the gastric wall hole 257, the outer end of introducer 226 may be connected to a suction source and a surgeon's index finger may cover the inlet opening 234, as suggested in FIG. 21. The open free end 232 of introducer 226 thus enables the flashback of bile, gastric juice or the like into the introducer to provide the surgeon with a visible indicator that the introducer is properly positioned in the lumina of the stomach. To facilitate emptying of a stomach, the cannulator 236 may be fitted onto the introducer 226 and slid down the introducer toward the free end to the extent indicated in FIG. 23 wherein the inlet opening 234 is covered by, but spaced from the cannulator, as also shown in section in FIG. 22. Thus an efficient “sump” is created. This sump suction allows rapid gastric emptying of vegetable matter, air, gastric secretions, inspissated mucous and/or other fluids (including those containing solids or particulates) without fowling of the suction system. Additionally, the introducer 226 may be reciprocated back and forth within the stomach to prevent clogging of the inlet opening 234. Thus the introducer 226 provides for rapid cannulation of the stomach in order to create a Stamm gastrostomy. It also provides for entry into the stomach without false passage within the layers of the gastric wall and without hemorrhage from the rich blood supply of the gastric wall. Finally, the introducer 226 provides for “clean” cannulation and decompression of the stomach without spillage of gastric contents.

As noted above, use of the introducer is preferred, but optional. As such, the cannulator 236 may be inserted or passed through the gastric wall after the hole or opening 234 therein is formed, with or without the use or assistance of the introducer 226. While it may be preferred to remove some or all of the stomach contents before continuing with the procedure, it is not necessary. Accordingly, in those procedures in which the stomach is to be emptied, at least in part, once the preferred level of stomach contents has been removed, the introducer 226 may be withdrawn from the stomach through the cannulator 236. In those procedures not involving or requiring dilation of the opening or hole in the abdominal or gastric walls of the patient and/or removal of some or all of the stomach contents, but in which it is elected to use an introducer 226, the introducer 226 may be withdrawn after the distal end of the cannulator 236 is in patient. Again, one skilled in the art will recognize that an introducer 226 is not required and that, when desired, a cannulator 236 may be inserted into the patient without the use of an introducer 226. In either instance, the cannulator 236 may then be manipulated by the surgeon externally of the stomach to move the inner end of the cannulator 236 through the pylorus. The feed tube or catheter 258, which was previously inserted through the abdominal wall as shown in FIG. 20, may then be passed within the cannulator 236 through the gastric wall 259 and pylorus 267 into the duodenum 270. The distal or free end 272 of the feed tube 258 is preferably soft and round so as not to pierce the retro-peritoneal duodenum as the feed tube is further advanced. On the other hand, the feed tube 258 should have sufficient column strength to follow around the curves of the duodenum. This is of note because one or more portions of the duodenum may be inaccessible to the surgeon's fingers as a result of the location of other organs in the patient.

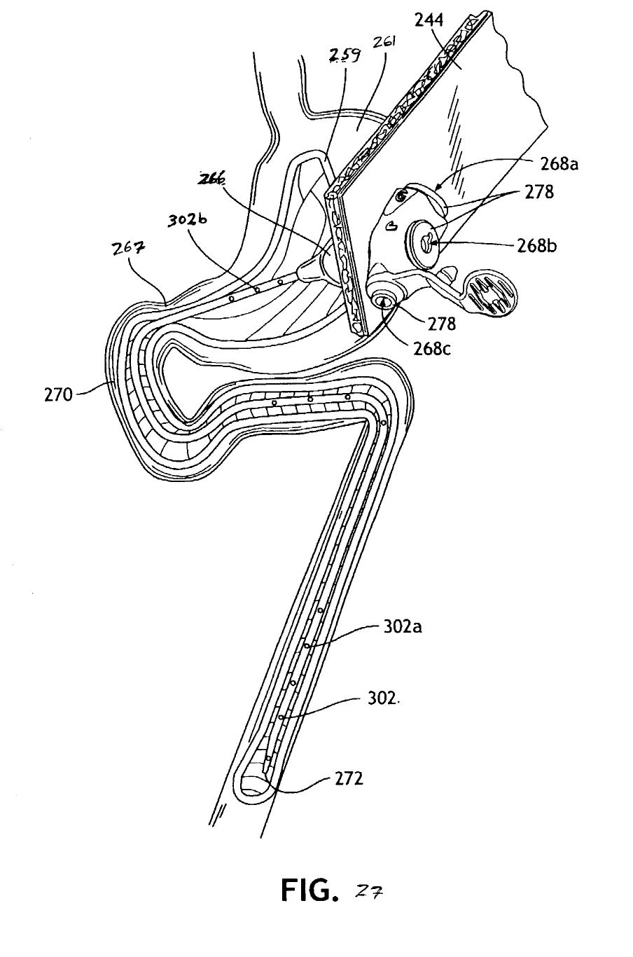

The distal end 272 of the feed tube 258 may continue to be advanced through the duodenum into the jejunum beyond the Ligament of Treitz as shown in FIGS. 25 and 26. At this position, the surgeon's fingers can pinch the jejunum to feel the position of the distal or free end of the feed tube 258 therein. Alternatively, and especially in those instances where the procedure is being performed in a non-surgical fashion or less invasive fashion, the feed tube 258 may contain a radiopaque material. The radiopaque material (shown, for example, in FIGS. 3A and 3B) is preferably located, at least in part, at or near the distal end of feed tube 258 so as to allow for a determination of the position of the distal end 272 of feed tube 258 or at least the location of the radiopaque material within the tube relative to the patient. The radiopaque material may be detected in any suitable manner known to those skilled in the art. One example of such a suitable detection means is the use of x-rays. The column strength of the tube is preferably increased as compared to other prior devices. As noted above, the column strength may be increased in a variety of suitable manners including, but not limited to, the selection of materials used to manufacture the tube or the presence of a radiopaque material in the catheter. The additional column strength and/or rigidity of the catheter not only assists with the placement of the catheter, but may also reduce the tendency of the jejunum to reflux or attempt to expel the catheter.

Upon proper placement of the feed tube 258, the cannulator 236 and/or guide wire (if present) is then withdrawn from the gastric wall hole 257 leaving the feed tube in place within the patient. Thus the cannulator 236 may enable rapid “blind” cannulation of the retro-peritoneal duodenum with passage of the jejunal tube beyond the Ligament of Treitz into the proximal jejunum. The primary purpose of the transpyloric cannulator is to provide passage of the transpyloric feed tube 258 with a particular emphasis on the prevention of laceration or perforation of the retro-peritoneal duodenum during this procedure.

Feed tube 258 terminates at its outer or upper end in a low profile head or fitting 262, described in more detail above. As one skilled in the art will appreciate based on the description of the feed tube or catheter 258 above, the retention mechanism, preferably a balloon or elongate sleeve 266, preferably will be advanced through the gastric wall hole 257 into the stomach during the above described procedure. In those embodiments having a inflatable balloon sleeve 266, a source of pressurized saline, for example, may be attached to the opening in the head of the device in communication with the balloon so that the balloon may be inflated as illustrated in FIG. 27. Desirably the balloon 266 is not inflated until after tube or catheter 258 is positioned in the preferred manner. While saline is a preferred fluid, any other suitable fluid (including, for example, air or distilled water) will work. One skilled in the art will appreciate that suitable fluids preferably will not degrade the catheter 258 or sleeve 266; however, if such a fluid does degrade the catheter or sleeve, the fluid is preferably one that will not adversely affect the patient if the patient were to be exposed thereto.

As will be appreciated from the detailed description above, one of the openings 268a in the head of the apparatus will be in communication with one or more apertures or holes 302b (shown in FIG. 27) through the side wall of the feed tube for aspirating the stomach when needed. While the apertures 302b may provide for natural (i.e. non-evacuative pressure relied, attachment of a vacuum source or an adapter connected to a vacuum source to opening 268a provides the opportunity for aspiration of fluids, including those containing solids, from the stomach when needed.

Another feature of the feed tube 258 of the present invention is that the length of the tube from the head or fitting 262 to the free end 272 of the tube 258 should be sufficient for placement of the free end in the jejunum beyond the Ligament of Treitz. The distal portion of the jejunal feed tube 258 will preferably have a radiopaque material within its wall to allow postoperative visualization on subsequent X-rays.

While one method of applying or positioning the devices of the present invention is discussed above, alternative methods for applying or positioning the devices include, for example, a method for placement of an elongated low profile flexible feed tube through the abdominal wall, gastric wall, pylorus and duodenum of a patient into the jejunum beyond the Ligament of Treitz. One alternate method includes providing an abdominal wall trocar, an elongated tubular sheath of a size and shape to closely conform to the trocar, and an elongated flexible feed tube, the elongate flexible feeding tube having a proximal and a distal end, and having a low profile head attached thereto; telescopically sliding the sheath onto the trocar; thrusting the trocar and sheath together through the abdominal wall to form a puncture wound therethrough; removing the trocar from the sheath; puncturing the gastric wall at a position for alignment with the puncture wound in the abdominal wall; inserting a guide wire having a proximal end and a distal end through the abdominal wall and the gastric wall such that the distal end of the guide wire passes through the pylorus of the patient; passing the catheter over the guide wire; withdrawing the guide wire; and withdrawing the sheath. It will be appreciated by those skilled in the art that the step of withdrawing the sheath may occur prior to or after the step of withdrawing the guide wire. For example, the sheath may be removed once the guide wire is positioned in the desired location within the patient before the catheter or feed tube is passed over the guide wire or the sheath may remain in place until later in the procedure. That is, the sheath may remain in place until the feed tube or catheter is positioned in the desired location within the patient and may be removed before or after the guide wire is removed. In those instances where the step of withdrawing the sheath occurs prior to the step of withdrawing the guide wire, the sheath will preferably be removed after the step of inserting the guide wire, but may be removed as late in the procedure as after the feed tube or catheter has been properly positioned within the patient.

It will be appreciated by those of skill in the art that the step of passing the catheter over the guide wire further includes passing the distal end of the catheter over the proximate end of the guide wire and advancing the distal end of the catheter through the stomach, pylorus and duodenum into the jejunum beyond the Ligament of Treitz.

The method may also include the step of inserting the feed tube through the sheath in the abdominal wall. The step of inserting the feed tube through the sheath in the abdominal wall preferably occurs between the steps of puncturing the gastric wall at a position for alignment with the puncture wound in the abdominal wall and the step of withdrawing the guide wire.

Further still the method may also include the steps of providing an elongated tubular gastrostomy introducer having a tapered free end with an inlet opening therethrough; inserting the gastrostomy introducer through the gastric wall puncture into the lumen of the stomach; and withdrawing the introducer. The step of inserting the gastrostomy introducer through the gastric wall puncture into the lumen of the stomach preferably occurs after the step of puncturing the gastric wall at a position for alignment with the puncture wound in the abdominal wall and prior to the step of inserting a guide wire having a proximal end and a distal end through the abdominal wall and the gastric wall such that the distal end of the guide wire passes through the pylorus of the patient. The method may further include the step of aspirating the stomach through the introducer. Preferably, the step of aspirating the stomach through the introducer occurs prior to the step of inserting a guide wire having a proximal end and a distal end through the abdominal wall and the gastric wall such that the distal end of the guide wire passes through the pylorus of the patient.

The method can further still include the steps of: providing an elongated slotted cannulator having a longitudinal slot and internal width to accommodate passage of the introducer therethrough; inserting the feed tube into the cannulator and advancing the distal end of the feed tube into the jejunum beyond the Ligament of Treitz; and withdrawing the cannulator through the puncture whereby the feed tube is maintained in its inserted position. The step of withdrawing the cannulator through the puncture such that the tube is maintained in its inserted position may preferably be achieved by peeling or otherwise removing the feed tube from the cannulator through the longitudinal slot therein. The cannulator is preferably of such a size and shape to allow for insertion thereof through the sheath.

Still further yet, the method may alternatively include the steps of providing an elongated slotted cannulator having a longitudinal slot and internal width to accommodate passage of the introducer therethrough; fitting the slotted cannulator onto the introducer and passing the cannulator through the gastric wall puncture; withdrawing the introducer through the cannulator; inserting the feed tube into the cannulator and advancing the distal end of the feed tube into the jejunum beyond the Ligament of Treitz; and withdrawing the cannulator through the puncture whereby the feed tube is maintained in its inserted position. As noted above, the step of withdrawing the cannulator through the puncture such that the tube is maintained in its inserted position may preferably be achieved by peeling or otherwise removing the feed tube from the cannulator through the longitudinal slot therein. The cannulator is preferably removed after the tube is positioned, however, in one or more embodiments, the cannulator may be removed prior to the insertion of or the passing of the tube or catheter into the patient.

Additionally, one skilled in the art will appreciate that fitting the cannulator onto the introducer may include, but is not limited to, fitting, snapping, placing, guiding and the like. Any and all other suitable techniques of positioning the cannulator onto, about or adjacent the introducer in accordance with the present invention are also contemplated and intended to be within the definition of the term “fitting” when used in context of the cannulator.

As noted above in connection with one of the other placement methods, one skilled in the art will recognize that the cannulator may be used even where an introducer is not. That is, the cannulator may be inserted or passed through the gastric wall after the hole or opening therein is formed, without the use or assistance of the introducer. While in some cases it may be preferred to enlarge the opening in the gastric wall and/or the abdominal wall and/or to remove some or all of the stomach contents before continuing with the procedure, it is not necessary. Accordingly, in those procedures in which the openings in the gastric and/or abdominal walls are to be enlarged the introducer may be withdrawn through the cannulator once the desired size openings have been created or established. Alternatively, where the stomach is to be emptied, at least in part, once the preferred level of stomach contents has been removed, the introducer may be withdrawn from the stomach through the cannulator. In those procedures not involving or requiring the removal of some or all of the stomach contents, the introducer may be withdrawn after the cannulator is in place. Again, one skilled in the art will recognize that an introducer is not required and that when desired a cannulator may be inserted into the patient without the use of an introducer. In either instance, the cannulator may then be manipulated by the surgeon externally of the stomach to move the distal end of the cannulator through the pylorus. The feed tube or catheter may then be passed within the cannulator through the gastric wall and pylorus into the duodenum. Alternatively, a guide wire (not shown) may be used in combination with or instead of the cannulator at this point in the procedure.

Each of the methods herein including a cannulator may also provide for the step of manipulating the cannulator externally of the stomach to move the distal end of the cannulator within the stomach through the pylorus of the patient. Preferably, the step of manipulating the cannulator occurs prior to the step of inserting the feed tube; however, it is contemplated that later manipulation of the cannulator may be needed to complete the positioning of the feed tube.

The present invention is also directed to a method for placement of a low profile feed tube through the abdominal wall, gastric wall, pylorus and duodenum of a patient into the jejunum beyond the Ligament of Treitz. The method includes the steps of providing a low profile feeding tube, and a guide wire; inserting the guide wire through the abdominal and gastric walls of the patient; positioning the guide wire in the patient beyond the Ligament of Treitz; passing the low profile tube over the guide wire such that the distal end of the feed tube is positioned beyond the Ligament of Treitz; and removing the guide wire. One skilled in the art will recognize that the guide wire may be passed through the abdominal wall of the patient either through a pre-existing hole, puncture, stoma or the like or a new opening may be created. Where the guide wire is to be inserted through a pre-existing opening or stoma, a pre-existing tube or catheter may be in place which needs to be removed. The removal of the pre-existing tube or catheter may occur prior to the insertion of the guide wire. Alternatively, the guide wire may be passed through the pre-existing tube before the tube is removed over the guide wire, leaving the guide wire in place, and then completing the placement method.

The catheter or feeding tube in the method described above may also have a retention means and the method may further include the step of activating or triggering the retention mechanism depending on the type of retention mechanism in use. The step of activating the retention mechanism should occur after the feed tube is positioned in the patient and may act to secure the tube within the patient. Prior to the insertion of the low profile tube, it is preferable to measure the stoma or opening in the abdominal and gastric walls of the patient to provide for the selection of the proper size low profile device. That is, unlike other pre-existing devices which use SECURE-LOK* rings (such as those available from the Applicants' Assignee) or the like to maintain positioning of the device, the current apparatus relies on the retention mechanism and the head of the device to maintain the positioning of the device. As such, failure to measure the stoma or opening and/or the failure to use a device with the proper sizing (i.e. distance between the low profile head and the retention mechanism) may result in a loose and undesirable fit, if the device is too large in this regard, or a device which pinches or squeezes the patient or which cannot activate or deploy its retention mechanism, if the device is too small in this regard. In accordance with the above, the methods of the present invention may further include the steps of measuring the stoma length and selecting the proper size low profile feed tube.

The guide wire mentioned above may be steerable such that the step of positioning the guide wire may further include maneuvering the guide wire into the desired position within the patient. Once the device has been positioned or placed in accordance with the present invention, it is preferable to verify the placement or positioning of the device and more specifically, the distal end thereof. Suitable methods include, for example, radiographic (e.g. x-ray) verification as discussed above.

Another method of the present invention directed to the placement of an elongated low profile feed tube through the abdominal wall, gastric wall, pylorus and duodenum of a patient into the jejunum beyond the Ligament of Treitz, includes providing a low profile feeding tube, a guide wire, and an endoscope, the endoscope having at least a working channel; inserting the endoscope through the patient's abdominal wall (e.g. through a pre-existing opening, hole, stoma or the like or through a newly created opening, hole or the like), gastric wall and past the Ligament of Treitz;

inserting the guide wire through the patient's abdominal wall, gastric wall and into the jejunum of the patient; removing the endoscope from the patient; passing the low profile feeding tube over the guide wire; and removing the guide wire. It is contemplated that the step of inserting the guide wire into the jejunum of the patient may further include inserting the guide wire into the endoscope and feeding the guide wire through the endoscope.

As noted above, the feed tube may include a retention means and the method may further include the step of activating the retention mechanism. The step of activating the retention mechanism preferably occurs after the step of passing the low profile feeding tube over the guide wire. In those instances where a tube is already in place in the patient (e.g. a temporary tube or one that needs to be replaced for some reason), the method may also include removing the existing tube from the abdominal wall of the patient. The existing tube may be removed from the patient prior to the insertion of the guide wire or the guide wire may be passed through the existing tube and then the existing tube may be removed. In those instances where the guide wire is passed through the existing tube, once the existing tube or catheter is removed, the endoscope may be passed over the guide wire to ensure that the wire is properly positioned before passing the catheter or tube over the wire.

An alternate method for placement of an elongated low profile catheter through the abdominal wall, gastric wall, pylorus and duodenum of a patient into the jejunum beyond the Ligament of Treitz includes the steps of providing a low profile catheter, a guide wire, and an endoscope, the endoscope having grasping means at the distal end thereof; inserting the endoscope through the patients mouth and into the patient's stomach; inserting the low profile catheter through the abdominal and gastric walls of the patient; grasping the catheter with the grasping means; positioning the catheter (and more specifically the distal end of the catheter) past the Ligament of Treitz with the endoscope; releasing the catheter from the grasping means; and removing the endoscope. The placement of the catheter may be verified visually (e.g. via the endoscope if markings are present on or along the catheter) or radiographically as discussed above. It is contemplated that the grasping means discussed in connection with the distal end of the endoscope includes both means which are or can be affixed to the distal end of the endoscope and means which may be passed through the endoscope so as to extend, at least in part, from the distal end thereof. Suitable grasping means include, for example, snares, forceps, and the like.

Yet another method for the placement of a low profile feeding tube into the jejunum of a patient and beyond the Ligament of Treitz includes providing a low profile feeding tube, a guide wire, and an endoscope, the endoscope having grasping means at the distal end thereof; inserting the endoscope through the patient's mouth and into the stomach; inserting the guide wire through the abdominal and gastric walls of the patient; grasping the guide wire with the grasping means; using the endoscope to position the guide wire, and more specifically the distal end of the guide wire, past the Ligament of Treitz; releasing the guide wire from the grasping means; removing the endoscope; passing the low profile feeding tube over the guide wire; and removing the guide wire. Preferably the feeding tube has a retention means. Where the tube or catheter has a retention means, the method may also include the step of activating the retention mechanism so as to secure the tube within the patient. The retention mechanism and the head of the device are preferably arranged or positioned to create a relatively close fit between the low profile head and the abdomen of the patient when the tube is properly positioned and the retention mechanism is activated or otherwise expanded. As noted above, this is best achieved where the method also includes the steps of measuring the stoma length and selecting the proper size catheter or feeding tube. Additionally, to achieve the preferred effects of the low profile device, the method may also include a step of verifying placement of the feeding tube within the patient. Placement verification is preferably done by way of radiographically, but may be performed in any other suitable manner as will be appreciate by those skilled in the art.

Lastly, a final alternate method for placing a low profile transjejunal feeding tube into the jejunum of a patient beyond the Ligament of Treitz includes providing a nasogastric tube, a plurality of fasteners, an introducer needle having a cannula, at least one guide wire having a proximal end and a distal end, and a feeding tube having a proximal end and a distal end, the proximal end of the tube having a low profile head; passing the nasogastric tube through the nose of the patient such that the nasogastric tube terminates in the stomach; insufflating the stomach via the nasogastric tube; fastening the stomach to the abdominal wall at a plurality of points surrounding a future stoma site; inserting the needle and cannula through the abdominal and gastric walls of the patent, so as to form a puncture or other opening; removing the needle; inserting the guide wire through the cannula; removing the cannula; passing the feeding tube over the guide wire such that the distal end of the feeding tube is located beyond the Ligament of Treitz; removing the guide wire; and removing the nasogastric tube. It will be appreciated that the nasogastric tube may be removed at any point in procedure after insufflation, but preferably the tube will not be removed until the stomach wall has been attached to the abdominal wall and more preferably not until the procedure is complete. It will also be appreciated that the needle (which may be, but is not limited to, a needle, an electro-cautery device or any other suitable device or means) may be used to form a puncture or any other opening in the abdominal or gastric wall which will enable the remainder of the method described herein. The fasteners used in the method above may be any fasteners suitable for the use described, but may include, for example, sutures or T-fasteners.

The method may also include the steps of providing a dilator; inserting the dilator; expanding the puncture or other opening; and removing dilator. The steps of inserting the dilator and removing the dilator will preferably occur after the step of removing the cannula and before the step of passing the feeding tube over the guide wire.

In some procedures, it will be preferable to use two guide wires, a short guide wire and a long guide wire. Where two wires are used the shorter wire will be inserted through the cannula as described above and left in place until the dilation steps are complete. The short wire is then removed and replaced with the longer guide wire. Preferably, the longer wire will be inserted such that the distal end of the guide wire is positioned beyond the Ligament of Treitz. This will enable the individual positioning the catheter or tube to more readily place the catheter or tube. The use of the different length wires is for convenience purposes and to facilitate the procedure, however one skilled in the art will recognize that the procedure only requires one guide wire.

Depending on the size of the feeding tube or catheter to be inserted, the opening in the patient's abdominal and gastric walls may have to be expanded different amounts to allow insertion of the tube or catheter. As such the method discussed herein may also provide for the provision of a plurality of dilators having different diameters. The dilators may be used serially, that is smallest to largest, until the preferred opening size is achieved. It will be appreciated that the size of the preferred opening may be larger than the size of the tube or catheter to be inserted so as to provide for easier insertion or passage of the tube, including any portions of the tube or catheter which may have a larger diameter (e.g. those portions of the tube or catheter about which the retention mechanism is affixed) through the opening. In order to radially expand the opening in a serial fashion, a first dilator having a diameter will be inserted into the opening, preferably over the guide wire, and then removed. A second dilator having a diameter larger than the first is then inserted and removed. The insertion and removal of dilators having a diameter larger than the one immediately before it may continue until the desired opening diameter is achieved. The opening or stoma size (i.e. the distance between the outer surface of the patients abdominal wall and the inside of the gastric wall at the point of the opening) is preferably measured (with, for example, a stoma measuring device as known to those skilled in the art) or otherwise determined (with, for example, a guide wire) and a proper size low profile tube should then be selected.

In one embodiment of the method discussed above, the last or largest dilator used may include a sheath over the exterior of the dilator. In use the sheath may be left in place when the dilator is removed. The guide wire (if not already inserted) and tube may then be passed through the sheath whereby the sheath may provide a retaining wall effect as related to the opening in the patients abdominal and gastric walls and may provide for easier passage of the tube into the patient as a less constrictive and/or tortuous path may be experienced. Where a sheath is used, the method of placing the low profile tube or catheter will further include further include the step of removing the sheath. The step of removing the sheath preferably occurs after the feeding tube is positioned within the patient. To enable removal of the sheath from the tube the sheath may be removed from the feeding tube, by, for example, peeling away or splitting or slicing the sheath and then pulling it away or otherwise removing it from the tube. It will be appreciated that the sheath may be removed just prior to final positioning of the tube as some space about the head of the device and the patient's abdomen will be necessary to enable the sheath's removal. Furthermore, final positioning of the sheath will generally not be possible until the sheath is removed as the sheath may obstruct the expansion of the retention mechanism.

Finally, while it is preferable for the low profile tube or catheter to be properly sized at the time it is placed, in some instances, initial placement of the tube or catheter (i.e. where a new or enlarged opening or stoma is created in the patient so as to allow the placement of the device) may cause some trauma to the abdominal wall and/or gastric wall such that swelling which is common with all such traumatic placements occurs. In such instances, any swelling that may occur will in most cases dissipate after a number of days. The reduction in swelling may result in the fit between the retention mechanism and the low profile head of the device and the patient to loosen. In such cases, as well as those cases where an improperly sized device has been used, a spacer may be provided and inserted between the abdomen of the patient the head of the feeding tube such that the retention mechanism and the head of the tube create a tighter or more secure fit with the patient. Although spacers may be used with the present invention, the presence of a small gap or separation between the head of the device and the abdominal wall of the patient after insertion of the device will not affect the function of the device so long as the distal end of the device remains properly positioned.

Whereas the invention has been shown and described in connection with preferred embodiments thereof, it is understood that many modifications, additions, and substitutions may be made which are within the intended broad scope of the appended claims.

We claim:

1. A method for placement of a low profile feed tube through the abdominal wall, gastric wall, pylorus and duodenum of a patient into the jejunum beyond the Ligament of Treitz, the method comprising the steps:
providing a low profile feeding tube, and a guide wire;
inserting the guide wire through the abdominal wall of the patient;
positioning the guide wire in the patient beyond the Ligament of Treitz;
passing the low profile feeding tube over the guide wire such that the distal end of the feed tube is positioned beyond the Ligament of Treitz; and
removing the guide wire.

2. The method of claim 1, wherein the feed tube has a retention means and wherein the method further comprises the step of activating the retention mechanism.

3. The method of claim 1 further comprising the steps of:
measuring the stoma length; and
selecting the proper size low profile feed tube.

4. The method of claim 1 further comprising the step of verifying placement of the feeding tube within the patient.

5. The method of claim 4, wherein the step of verifying placement of the feeding tube comprises radiographic verification.

6. The method of claim 1, wherein the guide wire is steerable, and wherein the step of positioning the guide wire may further comprise maneuvering the guide wire.

7. The method of claim 1 further comprising the step of removing an existing tube from the abdominal wall of the patient, wherein the step of inserting the guide wire occurs prior to the step of removing the existing tube from the patient, and wherein the guide wire is passed through the existing tube.

8. The method of claim 1 further comprising the step of removing an existing tube from the abdominal wall of the patient, wherein the step of removing the existing tube from the patient occurs before inserting the guide wire.

9. A method for placement of an elongated low profile feed tube through the abdominal wall, gastric wall, pylorus and duodenum of a patient into the jejunum beyond the Ligament of Treitz, the method comprising the steps:
providing a low profile feeding tube, a guide wire, and an endoscope, the endoscope having at least a working channel;
inserting the endoscope through the patients abdominal wall, gastric wall and past the Ligament of Treitz;
inserting the guide wire through the patient's abdominal wall, gastric wall and into the jejunum of the patient;
removing the endoscope from the patient;
passing the low profile feeding tube over the guide wire; and
removing the guide wire.

10. The method of claim 9, wherein the step of inserting the guide wire into the jejunum of the patient further comprises inserting the guide wire into the endoscope and feeding the guide wire through the endoscope.

11. The method of claim 9, wherein the feed tube has a retention means and wherein the method further comprises the step of activating the retention mechanism.

12. The method of claim 9 further comprising the step of verifying placement of the feeding tube within the patient.

13. The method of claim 9 further comprising the step of removing an existing tube from the patient, wherein the step of removing the existing tube from the patient occurs before inserting the guide wire.

14. The method of claim 9 further comprising the step of removing an existing tube from the patient, wherein the step of removing the existing tube from the patient occurs prior to the step of inserting the endoscope into the patient, and wherein the guide wire is passed through the existing tube.

15. A method for placement of an elongated low profile catheter through the abdominal wall, gastric wall, pylorus and duodenum of a patient into the jejunum beyond the Ligament of Treitz, the method comprising the steps:
providing a low profile catheter, a guide wire, and an endoscope, the endoscope having grasping means at the distal end thereof;
inserting the endoscope through the patients mouth and into the patient's stomach;
inserting the low profile catheter through the abdominal and gastric walls of the patient;
grasping the catheter with the grasping means;
positioning the catheter past the Ligament of Treitz with the endoscope;
releasing the catheter from the grasping means; and
removing the endoscope.

16. The method of claim 15 further comprising the step of verifying placement of the catheter within the patient.

17. The method of claim 16, wherein the step of verifying placement of the catheter within the patient is done visually or radiographically.

18. A method for placement of a low profile feeding tube through the abdominal wall, gastric wall, pylorus and duodenum of a patient into the jejunum beyond the Ligament of Treitz, the method comprising the steps:
providing a low profile feeding tube, a guide wire, and an endoscope, the endoscope having grasping means at the distal end thereof;
inserting the endoscope through the patients mouth and into the stomach;
inserting the guide wire through the abdominal and gastric walls of the patient;
grasping the guide wire with the grasping means;
positioning the guide wire past the Ligament of Treitz with the endoscope;
releasing the guide wire from the grasping means;
removing the endoscope;
passing the low profile feeding tube over the guide wire; and
removing the guide wire.

19. The method of claim 18, wherein the feeding tube has a retention means and wherein the method further comprises the step of activating the retention mechanism so as to secure the tube within the patient.

20. The method of claim 18 further comprising the steps of:
measuring the stoma length; and
selecting the proper size low profile feeding tube.

21. The method of claim 18 further comprising the step of verifying placement of the feeding tube within the patient.

22. The method of claim 21, wherein the step of verifying placement of the feeding tube comprises radiographic verification.

23. A method for placing a low profile transjejunal feeding tube into the jejunum of a patient beyond the Ligament of Treitz, the method comprising the steps:
providing a nasogastric tube, a plurality of T-fasteners, an introducer needle having a cannula, at least one a guide wire having a proximal end and a distal end, a dilator, and a feeding tube having a proximal end and a distal end, the proximal end of the tube having a low profile head;
passing the nasogastric tube through the nose of the patient such that the nasogastric tube terminates in the stomach;
insufflating the stomach;
fastening the stomach to the abdominal wall at a plurality of points surrounding a future stoma site;
inserting the needle and cannula through the abdominal and gastric wall of the patient, so as to form a puncture;
removing the needle;
inserting the guide wire through the cannula;
removing the cannula;
inserting the dilator;
expanding the puncture;
removing dilator;
inserting guide wire;
passing the feeding tube over the guide wire such that the distal end of the feeding tube is located beyond the Ligament of Treitz;
removing the guide wire; and
removing the nasogastric tube.

24. The method of claim 23 further comprises providing a plurality of dilators having different diameters and radially expanding the puncture by inserting a first dilator having a diameter, removing the first dilator, inserting a second dilator having a diameter larger than the first and removing the second dilator.

25. The method of claim 23, wherein the dilator further comprises a sheath over the exterior of the dilator, wherein the sheath is left in place when the dilator is removed and the guide wire and tube are passed through the sheath.

26. The method of claim 25 further comprising the step of removing the sheath, wherein the step of removing the sheath occurs after the feeding tube is positioned within the patient.

27. The method of claim 26, wherein the sheath is peeled away from the feeding tube.

28. The method of claim 23, wherein the feeding tube has a retention means and wherein the method further comprises the step of activating the retention mechanism, wherein the step of activating the retention mechanism occurs after the distal end of the feeding tube is located beyond the Ligament of Treitz.

29. The method of claim 23 further comprising the steps of:
measuring the stoma length; and
selecting the proper size low profile feeding tube.

30. The method of claim 23 further comprising the step of verifying placement of the feeding tube within the patient.

31. The method of claim 23 further comprising the steps of:
providing a spacer;
inserting the spacer between the abdomen of the patient the head of the feeding tube.
